(19) United States
(12) Patent Application Publication
(10) Pub. No.: US 2024/0400558 A1
(43) Pub. Date: Dec. 5, 2024

(54) PROCESSES AND INTERMEDIATES FOR SYNTHESIS OF ADAGRASIB

(71) Applicant: MIRATI THERAPEUTICS, INC., San Diego, CA (US)

(72) Inventors: Thomas Scattolin, San Diego, CA (US); Cheng Chen, San Diego, CA (US); Tawfik Gharbaoui, San Diego, CA (US); Yonghong Gan, San Diego, CA (US); Michal Achmatowicz, San Diego, CA (US); David Snead, San Diego, CA (US)

(21) Appl. No.: 18/688,995

(22) PCT Filed: Sep. 8, 2022
(86) PCT No.: PCT/US2022/042835

§ 371 Date: Mar. 4, 2024

Related U.S. Application Data
(60) Provisional application No. 63/357,600, filed on Jun. 30, 2022.
(60) Provisional application No. 63/242,370, filed on Sep. 9, 2021.

(51) Int. Cl. C07D 471/04; B01J 31/22; B01J 31/24

(52) U.S. Cl. CPC C07D 471/04; B01J 31/2291; B01J 31/2409; B01J 2231/40; B01J 2231/763; B01J 2531/824

(57) ABSTRACT

The present invention relates to improved synthetic routes of synthesizing adagrasib. The invention also provides intermediates used in the provided synthetic routes.

### FIELD OF THE INVENTION

The present invention relates to improved synthetic routes for synthesis of adagrasib.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer et al., (2013) Current Opin Pharmcol. 13:394-401).

The role of activated KRas in malignancy was observed over thirty years ago (e.g., see Der et al., (1982) Proc. Natl Acad. Sci. USA 79(11):3637-3640). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma, with a G12C transversion being the most common activating mutation (e.g., see Dogan et al., (2012) Clin Cancer Res. 18(22):6169-6177, published online 2012 Sep. 26. doi: 10.1158/1078-0432.CCR-11-3265).

The well-known role of KRas in malignancy and the discovery of these frequent mutations in KRas in various tumor types made KRas a highly attractable target of the pharmaceutical industry for cancer therapy. Notwithstanding thirty years of large scale discovery efforts to develop inhibitors of KRas for treating cancer, no KRas inhibitor has demonstrated sufficient safety and/or efficacy to obtain regulatory approval (e.g., see McCormick (2015) Clin Cancer Res. 21 (8):1797-1801).

KRas G12C inhibitor compound 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (also known as MRTX849, and also known as adagrasib) has the following structure:

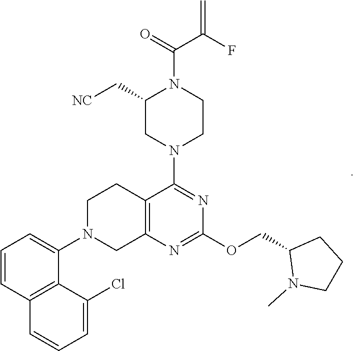 [SMILES: C=C(F)C(=O)N1CCN(c2nc(OC[C@@H]3CCCN3C)nc3c2CCN(c2cccc4cccc(Cl)c24)C3)C[C@@H]1CC#N ; "."]

Adagrasib is described, for example, in Example 478 of PCT Application WO 2019/099524.

While WO 2019/099524 describes methods of making adagrasib, there is a need in the art for improved synthetic routes of making adagrasib.

### SUMMARY OF THE INVENTION

The present invention, in one aspect, provides improved methods of making adagrasib.

In one aspect, the invention provides a method of synthesizing adagrasib, comprising the step of:
a) reacting a compound of the following structure:

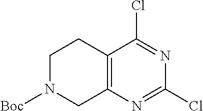 [SMILES: CC(C)(C)OC(=O)N1CCc2c(Cl)nc(Cl)nc2C1]

with a free base or a salt of a compound of the following structure:

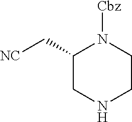 [SMILES: N#CC[C@H]1CNCCN1C(=O)OCc1ccccc1]

in the presence of a polar aprotic solvent and a base to produce a final compound of step (a) with the following structure:

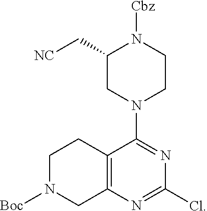 [SMILES: CC(C)(C)OC(=O)N1CCc2c(nc(Cl)nc2N2CCN(C(=O)OCc3ccccc3)[C@@H](CC#N)C2)C1]

As an alternative to the step (a), the method of the invention comprises step (a′):
a′) reacting a compound of the following structure:

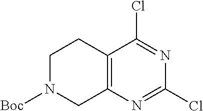 [SMILES: CC(C)(C)OC(=O)N1CCc2c(Cl)nc(Cl)nc2C1]

with the compound of the following structure:

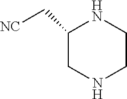

in the presence of a polar aprotic solvent and a base to produce the compound of the following structure:

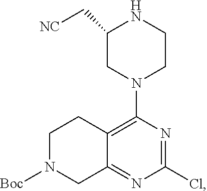 [SMILES: CC(C)(C)OC(=O)N1CCc2c(nc(Cl)nc2N2CCN[C@@H](CC#N)C2)C1]

and then
reacting this compound with benzyl chloroformate in the presence of sodium carbonate, methyl tert-butyl ether (MTBE) and water to produce a final compound of step (a′) with the following structure:

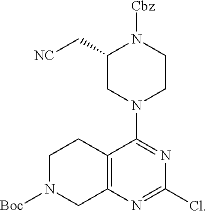 [SMILES: CC(C)(C)OC(=O)N1CCc2c(nc(Cl)nc2N2CCN(C(=O)OCc3ccccc3)[C@@H](CC#N)C2)C1]

In one aspect, the method of the invention further comprises step (b):
b) reacting the final compound of step (a) or step (a′) with (S)-(1-methylpyrrolidin-2-yl)methanol in the presence of a palladium catalyst, a base, a phosphorous-based ligand, and an aprotic solvent to produce a final compound of step (b) with the following structure:

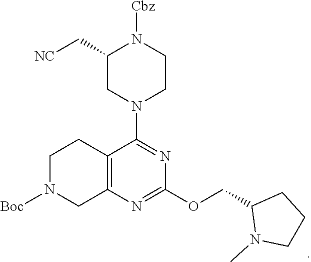

In another aspect, the method of the invention further comprises step (c):
c) reacting the final compound of step (b) with an acid to remove a Boc protecting group from the final compound of step (b) to produce a salt or free base of a final compound of step (c) with the following structure:

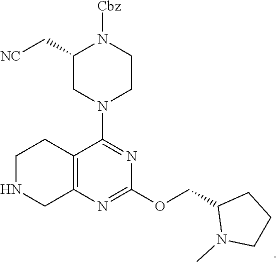

In one aspect, the method of the invention further comprises step (d):
d) reacting the salt or free base of the final product of step (c) with

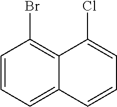 [SMILES: Clc1cccc2cccc(Br)c12]

in the presence of a palladium catalyst, a base, a phosphorous-based ligand, and an aprotic solvent to produce a final compound of step (d) with the following structure:

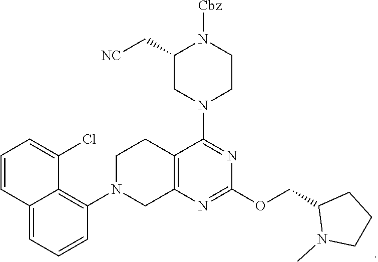

In one aspect, the method of the invention further comprises step (e):
e) reacting the final compound of step (d) with p-toluenesulfonic acid in the presence of a first solvent and an anti-solvent to produce a final compound of step (e) with the following structure:

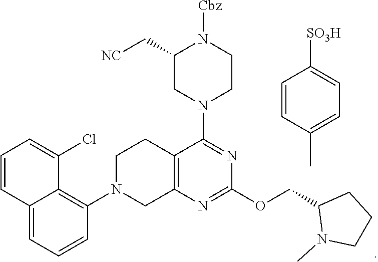

In one aspect, the method of the invention further comprises step (f):
f) reacting the final compound of step (e) with a thiol or thiolate in the presence of a base and a polar aprotic solvent to produce a final compound of step (f) with the following structure:

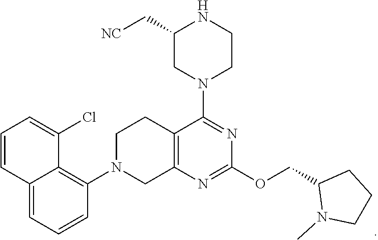

In one aspect, the method of the invention further comprises step (g):
(g) reacting the final compound of step (f) with 2-fluoroacrylic acid (or corresponding alkali or metal salts) and a coupling agent in the presence of a solvent and, optionally, a base to produce adagrasib.

In one aspect, the invention provides a method of synthesizing adagrasib, comprising the step of reacting

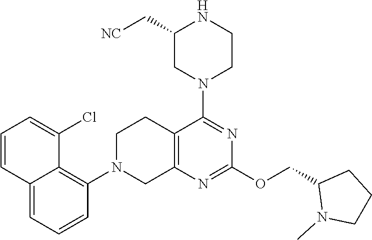

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting

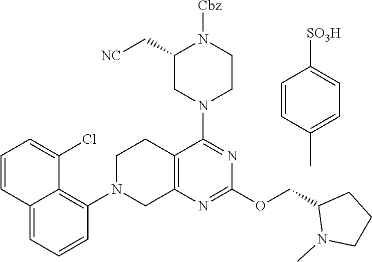

with a thiol or thiolate in the presence of a base and a polar aprotic solvent to produce

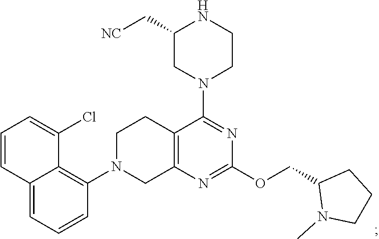

and
reacting

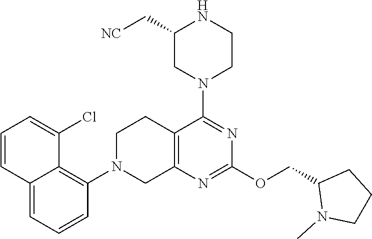

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting

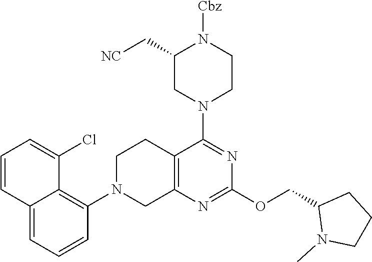

with p-toluenesulfonic acid in the presence of a first solvent and an anti-solvent to produce

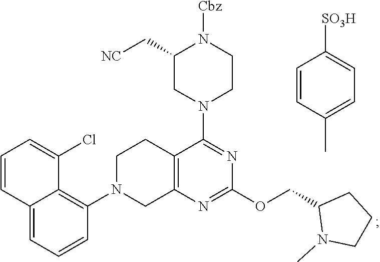

reacting

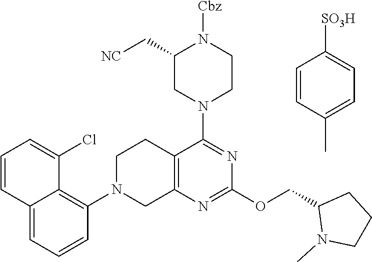

with a thiol or thiolate in the presence of a base and a polar aprotic solvent to produce

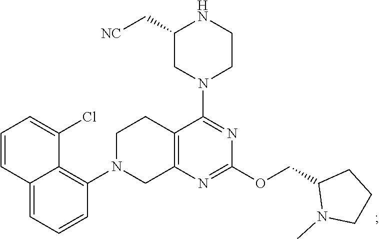

and
reacting

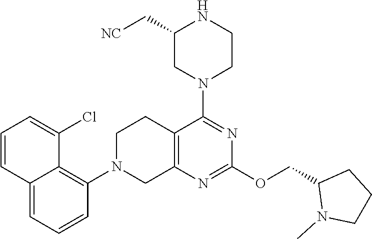

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting the free base of

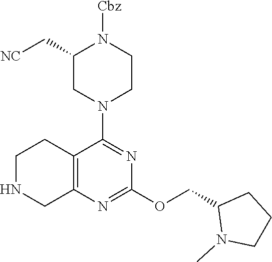

with

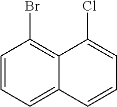 [SMILES: Clc1cccc2cccc(Br)c12]

in the presence of a palladium catalyst, a base, a phosphorous-based ligand, and an aprotic solvent to produce

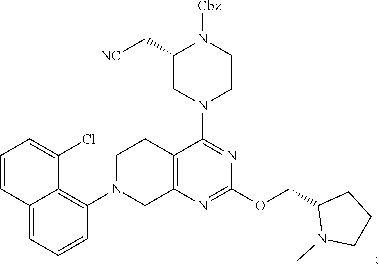

reacting

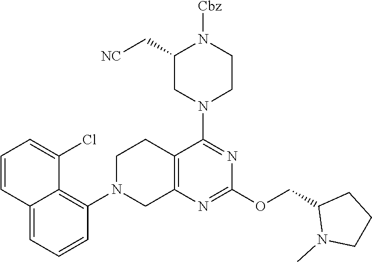

with p-toluenesulfonic acid in the presence of a first solvent and an anti-solvent to produce

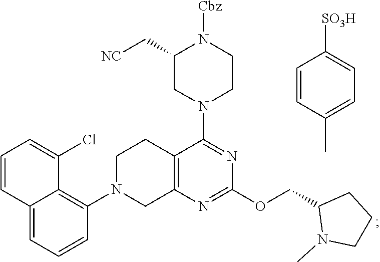

reacting

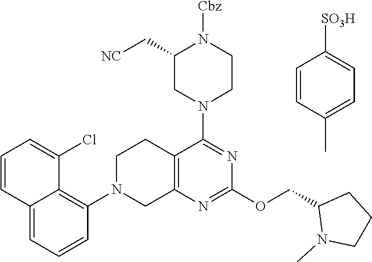

with a thiol or thiolate in the presence of a base and a polar aprotic solvent to produce

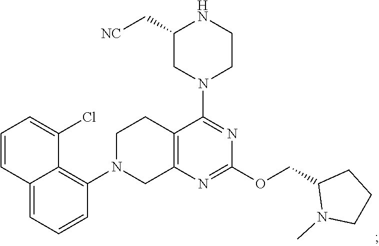

and
reacting

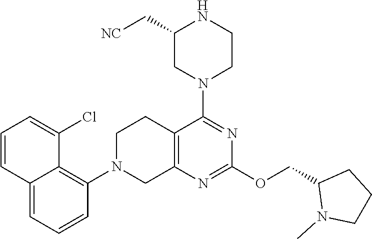

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting

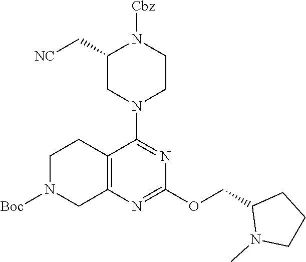

with an acid to remove a Boc protecting group to produce a salt or free base of

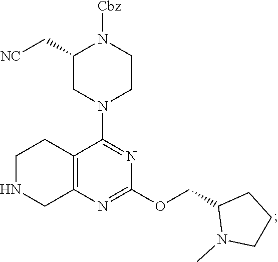

reacting the salt or free base of

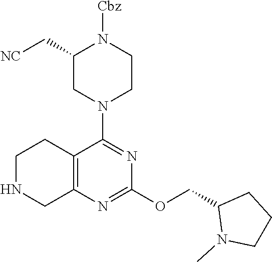

with

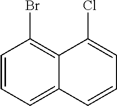 [SMILES: Clc1cccc2cccc(Br)c12]

in the presence of a palladium catalyst, a base, a phosphorous-based ligand, and an aprotic solvent to produce

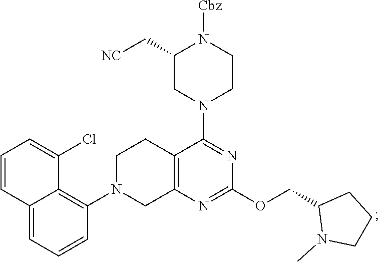

reacting

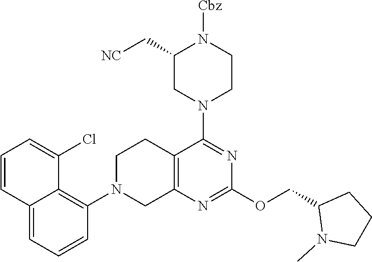

with p-toluenesulfonic acid in the presence of a first solvent and an anti-solvent to produce

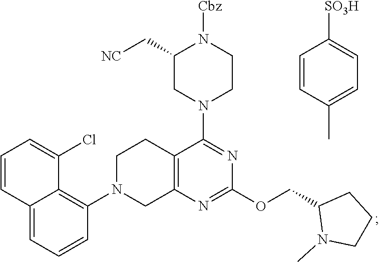

reacting

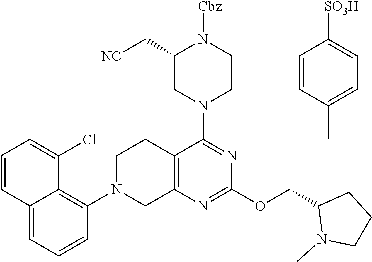

with a thiol or thiolate in the presence of a base and a polar aprotic solvent to produce

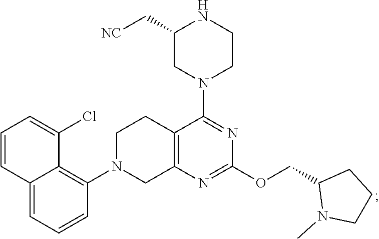

and
reacting

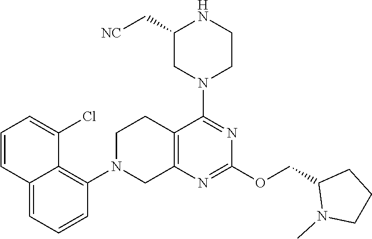

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting

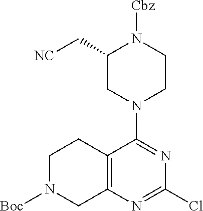 [SMILES: CC(C)(C)OC(=O)N1CCc2c(nc(Cl)nc2N2CCN(C(=O)OCc3ccccc3)[C@@H](CC#N)C2)C1]

with (S)-(1-methylpyrrolidin-2-yl)methanol in the presence of a palladium catalyst, a base, a phosphorous-based ligand, and an aprotic solvent to produce:

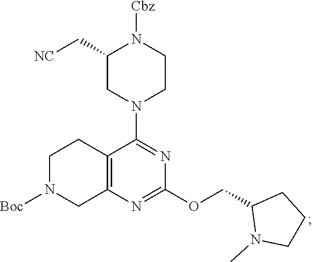

reacting

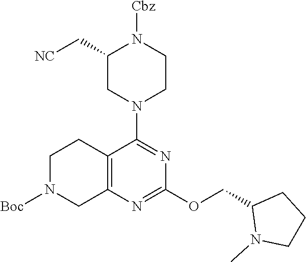

with an acid to remove a Boc protecting group to produce a salt or free base of:

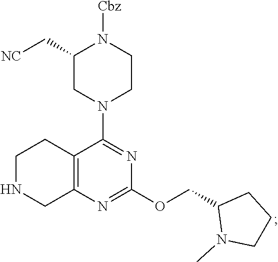

reacting the salt or free base of

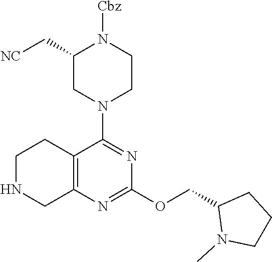

with

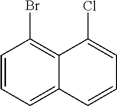 [SMILES: Clc1cccc2cccc(Br)c12]

in the presence of a palladium catalyst, a base, a phosphorous-based ligand, and an aprotic solvent to produce

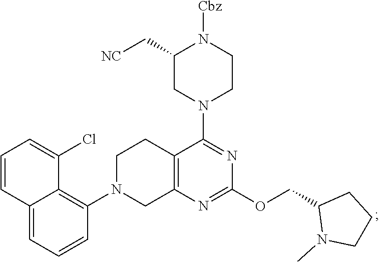

reacting

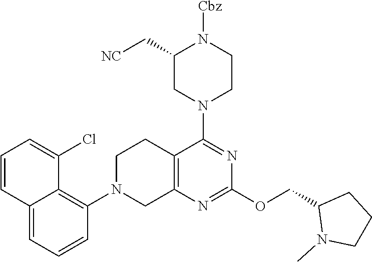

with p-toluenesulfonic acid in the presence of a first solvent and an anti-solvent to produce

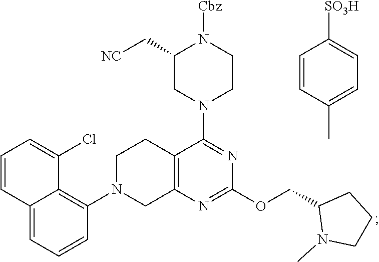

reacting

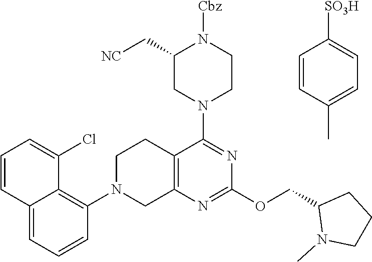

with a thiol or thiolate in the presence of a base and a polar aprotic solvent to produce

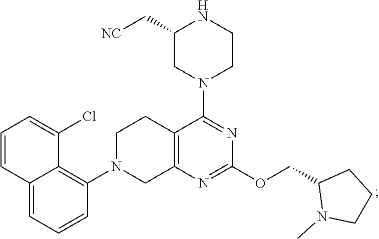

and
reacting

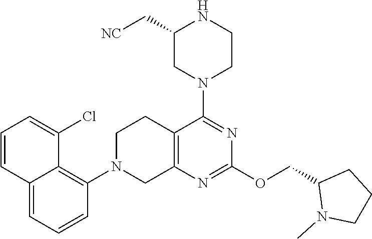

with 2-fluoroacrylic acid or alkali or metal salts of 2-fluoroacrylic acid with a coupling agent in the presence of a solvent and, optionally, a base to produce adagrasib.

In one aspect, the invention provides a method of synthesizing adagrasib, comprising the steps of:
a) reacting a compound of the following structure:

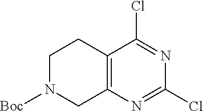 [SMILES: CC(C)(C)OC(=O)N1CCc2c(Cl)nc(Cl)nc2C1]

with a free base or salt of a compound of the following structure:

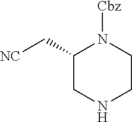 [SMILES: N#CC[C@H]1CNCCN1C(=O)OCc1ccccc1]

in the presence of a polar aprotic solvent and a base to produce a final compound of step (a) with the following structure:

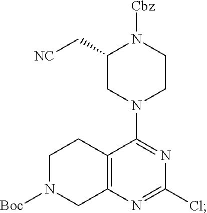 [SMILES: CC(C)(C)OC(=O)N1CCc2c(nc(Cl)nc2N2CCN(C(=O)OCc3ccccc3)[C@@H](CC#N)C2)C1]

b) reacting the final compound of step (a) with (S)-(1-methylpyrrolidin-2-yl)methanol in the presence of a palladium catalyst, a base, a phosphorous-based ligand, and an aprotic solvent to produce a final compound of step (b) with the following structure:

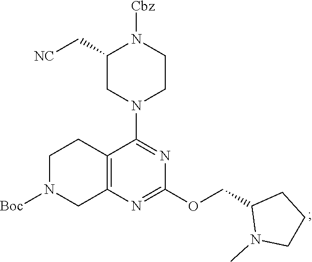

c) reacting the final compound of step (b) with an acid to remove a Boc protecting group from the final compound of step (b) to produce a salt or free base of a final compound of step (c) with the following structure:

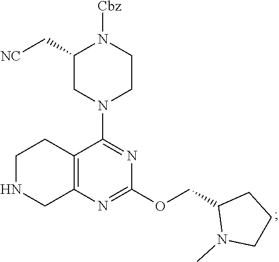

d) reacting the salt or free base of the final product of step (c) with

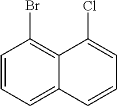 [SMILES: Clc1cccc2cccc(Br)c12]

in the presence of a palladium catalyst, a base, a phosphorous-based ligand, and an aprotic solvent to produce a final compound of step (d) with the following structure:

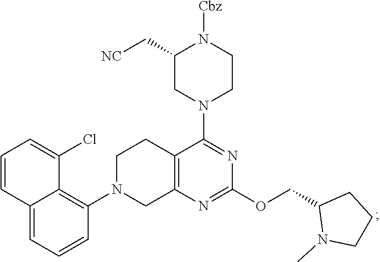

e) reacting the final compound of step (d) with p-toluenesulfonic acid in the presence of a first solvent and an anti-solvent to produce a final compound of step (e) with the following structure:

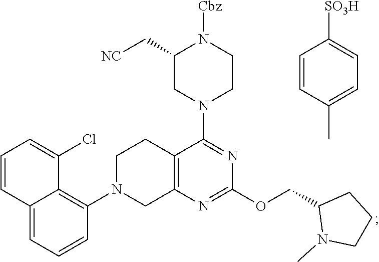

f) reacting the final compound of step (e) with a thiol or thiolate in the presence of a base and a polar aprotic solvent to produce a final compound of step (f) with the following structure:

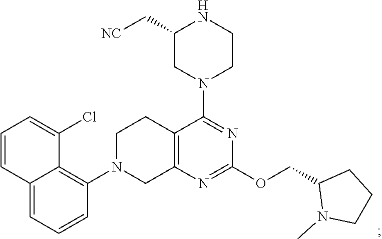

and
g) reacting the final compound of step (f) with 2-fluoroacrylic acid (or corresponding alkali or metal salts) and a coupling agent in the presence of a solvent and, optionally, a base to produce adagrasib.

In one aspect, the invention provides a method of synthesizing adagrasib, comprising the steps of:
a′) reacting a compound of the following structure:

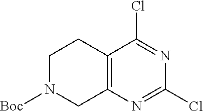 [SMILES: CC(C)(C)OC(=O)N1CCc2c(Cl)nc(Cl)nc2C1]

with the compound of the following structure:

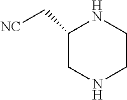

in the presence of a polar aprotic solvent and a base to produce the compound of the following structure:

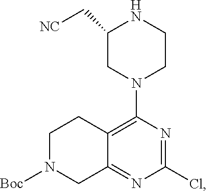 [SMILES: CC(C)(C)OC(=O)N1CCc2c(nc(Cl)nc2N2CCN[C@@H](CC#N)C2)C1]

and then
reacting this compound with benzyl chloroformate in the presence of sodium carbonate, methyl tert-butyl ether (MTBE) and water to produce a final compound of step (a′) with the following structure:

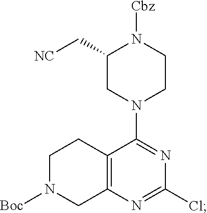 [SMILES: CC(C)(C)OC(=O)N1CCc2c(nc(Cl)nc2N2CCN(C(=O)OCc3ccccc3)[C@@H](CC#N)C2)C1]

reacting the final compound of step (a′) with (S)-(1-methylpyrrolidin-2-yl)methanol in the presence of a palladium catalyst, a base, a phosphorous-based ligand, and an aprotic solvent to produce a final compound of step (b) with the following structure:

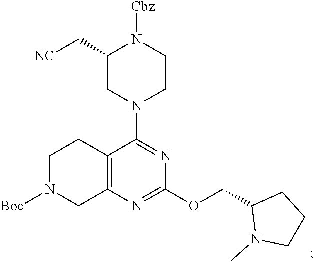

reacting the final compound of step (b) with an acid to remove a Boc protecting group from the final compound of step (b) to produce a salt or free base of a final compound of step (c) with the following structure:

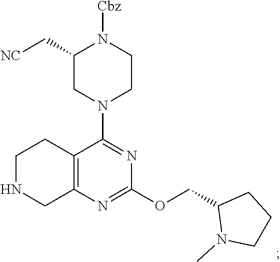

reacting the salt or free base of the final product of step (c) with

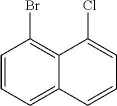 [SMILES: Clc1cccc2cccc(Br)c12]

in the presence of a palladium catalyst, a base, a phosphorous-based ligand, and an aprotic solvent to produce a final compound of step (d) with the following structure:

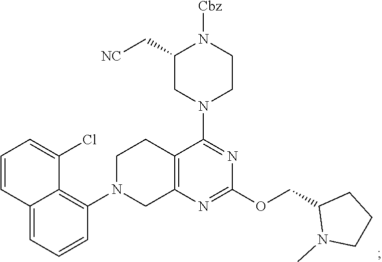

reacting the final compound of step (d) with p-toluenesulfonic acid in the presence of a first solvent and an anti-solvent to produce a final compound of step (e) with the following structure:

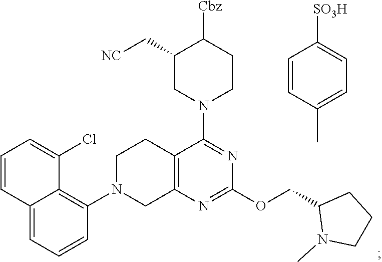

reacting the final compound of step (e) with a thiol or thiolate in the presence of a base and a polar aprotic solvent to produce a final compound of step (f) with the following structure:

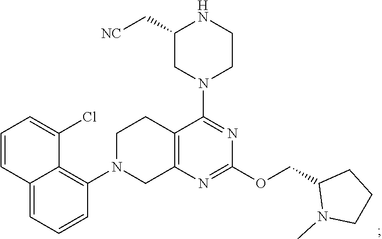

and
reacting the final compound of step (f) with 2-fluoroacrylic acid (or corresponding alkali or metal salts) and a coupling agent in the presence of a solvent and, optionally, a base to produce adagrasib.

In another aspect, the invention provides novel intermediate compounds of steps (a) through (e), such as:

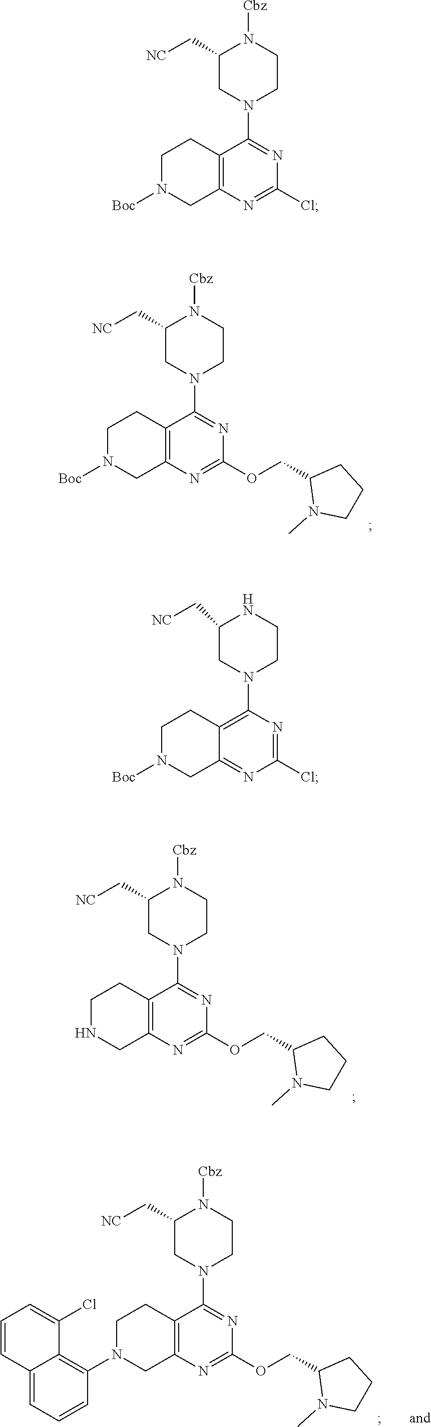

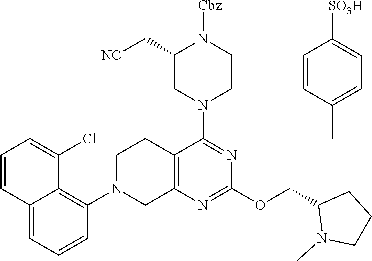

### BRIEF DESCRIPTION OF THE DRAWINGS

FIG. 1A is an image of phosphate particle sizes when procedures in Example 2 steps (b) and (c) are used;

FIG. 1B is an image of phosphate particle sizes when procedures in Example 2 steps (b′) and (c′) are used.

### DETAILED DESCRIPTION OF THE INVENTION

The present invention relates to improved synthetic routes for synthesizing adagrasib, as well as to novel intermediates used in the provided routes.

Although there is a known method of synthesizing adagrasib (see WO 2019/099524), the synthesis provided by the present invention is much improved, in that it has fewer steps, provides a higher isolated yield and a higher purity overall.

Furthermore, in the known synthesis, palladium-catalyzed hydrogenation led to the formation of side-products. The formation of these side-products is suppressed in the improved process. The use of a non-proprietary ligand in both palladium catalyzed steps, especially in the key C—N bond forming reaction with 1-bromo-8-chloronaphtalene significantly reduces the production cost.

The yield of the new synthesis has dramatically been improved over the previous synthesis, in part due to the increase in the yield of the final coupling step (from 47% to ca. 90% yield). This is believed to be due to using a more stable reagent, sodium 2-fluoroacrylate, and optimized reaction conditions.

The overall yield of adagrasib increased at least five-fold (<5% to 25%) using the described process and was demonstrated on multi-kilo scale (10-100 kg) with a shorter cycle time. In contrast, the previous synthesis was not readily amenable to scale-up.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Gly12Cys.

A “KRas G12C-associated disease or disorder” as used herein refers to diseases or disorders associated with or mediated by or having a KRas G12C mutation. A non-limiting example of a KRas G12C-associated disease or disorder is a KRas G12C-associated cancer.

As used herein, the term “adagrasib” refers to the compound which has the name: 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (also known as MRTX849) and which has the following structure:

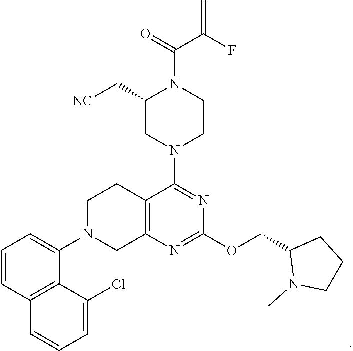

Adagrasib is described, for example, in Example 478 of PCT Application WO 2019/099524.

The term “adagrasib” encompasses all chiral (enantiomeric and diastereomeric) and racemic forms of the compound.

In one embodiment, the term “adagrasib” includes salts of the above compound, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Whenever the application refers to a chemical compound, unless specifically stated otherwise, the compound encompasses all chiral (enantiomeric and diastereomeric) and racemic forms of the compound.

The term “alkyl” is intended to mean a straight chain or branched aliphatic group having from 1 to 12 carbon atoms, alternatively 1-8 carbon atoms, and alternatively 1-6 carbon atoms. Other examples of alkyl groups have from 2 to 12 carbon atoms, alternatively 2-8 carbon atoms and alternatively 2-6 carbon atoms. Examples of alkyl groups include, without limitation, methyl, ethyl, propyl, isopropyl, butyl, isobutyl, sec-butyl, tert-butyl, pentyl, hexyl and the like. A “C0” alkyl (as in “C0-C3alkyl”) is a covalent bond.

The term “alkenyl” is intended to mean an unsaturated straight chain or branched aliphatic group with one or more carbon-carbon double bonds, having from 2 to 12 carbon atoms, alternatively 2-8 carbon atoms, and alternatively 2-6 carbon atoms. Examples of alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, alternatively 2-8 carbon atoms, and alternatively 2-6 carbon atoms. Examples of alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Examples of alkylene groups include, without limitation, methylene, ethylene, propylene, and butylene. Examples of alkenylene groups include, without limitation, ethenylene, propenylene, and butenylene. Examples of alkynylene groups include, without limitation, ethynylene, propynylene, and butynylene.

The term “carbocycle” as employed herein is intended to mean a cycloalkyl or aryl moiety.

The term “cycloalkyl” is intended to mean a saturated or unsaturated mono-, bi-, tri- or poly-cyclic hydrocarbon group having about 3 to 15 carbons, alternatively having 3 to 12 carbons, alternatively 3 to 8 carbons, alternatively 3 to 6 carbons, and alternatively 5 or 6 carbons. In certain embodiments, the cycloalkyl group is fused to an aryl, heteroaryl or heterocyclic group. Examples of cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated or unsaturated, straight chain or branched aliphatic group, wherein one or more carbon atoms in the group are independently replaced by a heteroatom selected from the group consisting of O, S, and N.

The term “aryl” is intended to mean a mono-, bi-, tri- or polycyclic aromatic moiety, for example a C6-C14aromatic moiety, for example comprising one to three aromatic rings. Alternatively, the aryl group is a C6-C10aryl group, alternatively a C6aryl group. Examples of aryl groups include, without limitation, phenyl, naphthyl, anthracenyl, and fluorenyl.

The terms “aralkyl” or “arylalkyl” are intended to mean a group comprising an aryl group covalently linked to an alkyl group. If an aralkyl group is described as “optionally substituted”, it is intended that either or both of the aryl and alkyl moieties may independently be optionally substituted or unsubstituted. Alternatively, the aralkyl group is (C1-C6)alk(C6-C10)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

As used herein, the term “pharmaceutically acceptable salt” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

As used herein, the term “mineral acid” (or “inorganic acid”) refers to any acid derived from an inorganic compound that dissociates to produce hydrogen ions (H+) in water. Nonlimiting examples of mineral acids include hydrochloric acid, nitric acid, phosphoric acid, sulfuric acid, boric acid, hydrofluoric acid, hydrobromic acid, perchloric acid, and hydroiodic acid.

As used herein, the term “organic acid” refers to any organic compound with acidic properties. Nonlimiting examples of organic acids include lactic acid, acetic acid, formic acid, citric acid, oxalic acid, uric acid, malic acid, and tartaric acid.

### Synthetic Schemes

In one aspect, the invention provides a method of synthesizing adagrasib, comprising the steps of:
a) reacting a compound of the following structure:

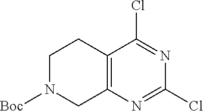 [SMILES: CC(C)(C)OC(=O)N1CCc2c(Cl)nc(Cl)nc2C1]

with a free base or a salt of a compound of the following structure:

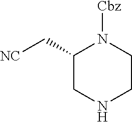 [SMILES: N#CC[C@H]1CNCCN1C(=O)OCc1ccccc1]

in the presence of a polar aprotic solvent and a base to produce a final compound of step (a) with the following structure:

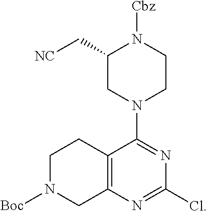 [SMILES: CC(C)(C)OC(=O)N1CCc2c(nc(Cl)nc2N2CCN(C(=O)OCc3ccccc3)[C@@H](CC#N)C2)C1]

As an alternative to the step (a), the method of the invention comprises step (a′):
a′) reacting a compound of the following structure:

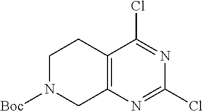 [SMILES: CC(C)(C)OC(=O)N1CCc2c(Cl)nc(Cl)nc2C1]

with the compound of the following structure:

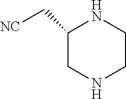

in the presence of a polar aprotic solvent and a base to produce the compound of the following structure:

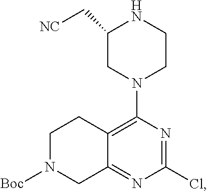 [SMILES: CC(C)(C)OC(=O)N1CCc2c(nc(Cl)nc2N2CCN[C@@H](CC#N)C2)C1]

and then
reacting this compound with benzyl chloroformate in the presence of sodium carbonate, methyl tert-butyl ether (MTBE) and water to produce a final compound of step (a′) with the following structure:

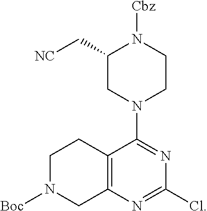 [SMILES: CC(C)(C)OC(=O)N1CCc2c(nc(Cl)nc2N2CCN(C(=O)OCc3ccccc3)[C@@H](CC#N)C2)C1]

In one aspect, the method of the invention further comprises step (b):
b) reacting the final compound of step (a) or step (a′) with (S)-(1-methylpyrrolidin-2-yl)methanol in the presence of a palladium catalyst, a base, a phosphorous-based ligand, and an aprotic solvent to produce a final compound of step (b) with the following structure:

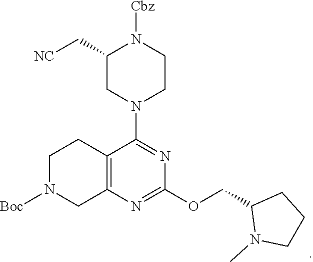

In another aspect, the method of the invention further comprises step (c):
c) reacting the final compound of step (b) with an acid to remove a Boc protecting group from the final compound of step (b) to produce a salt or free base of a final compound of step (c) with the following structure:

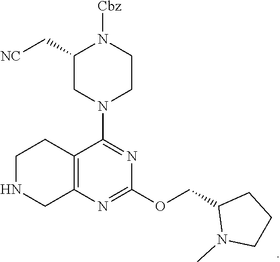

In one aspect, the method of the invention further comprises step (d):

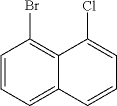 [SMILES: Clc1cccc2cccc(Br)c12]

d) reacting the salt or free base of the final product of step (c) with in the presence of a palladium catalyst, a base, a phosphorous-based ligand, and an aprotic solvent to produce a final compound of step (d) with the following structure:

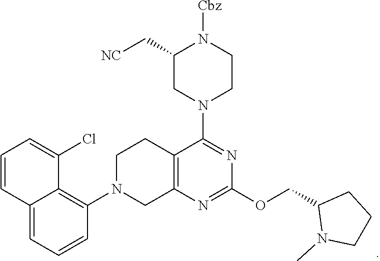

In one aspect, the method of the invention further comprises step (e):
e) reacting the final compound of step (d) with p-toluenesulfonic acid in the presence of a first solvent and an anti-solvent to produce a final compound of step (e) with the following structure:

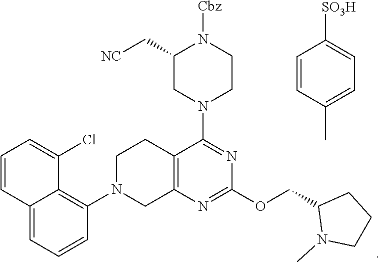

In one aspect, the method of the invention further comprises step (f):
f) reacting the final compound of step (e) with a thiol or thiolate in the presence of a base and a polar aprotic solvent to produce a final compound of step (f) with the following structure:

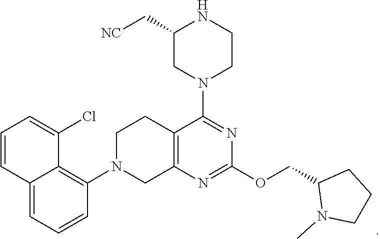

In one aspect, the method of the invention further comprises step (g):
(g) reacting the final compound of step (f) with 2-fluoroacrylic acid (or corresponding alkali and metal salts) and a coupling agent in the presence of a solvent and, optionally, a base to produce adagrasib.

In one aspect, the invention provides a method of synthesizing adagrasib, comprising the step of reacting

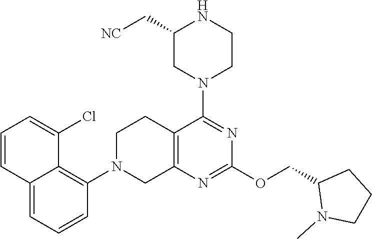

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting

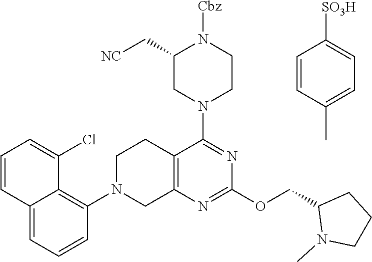

with a thiol or thiolate in the presence of a base and a polar aprotic solvent to produce

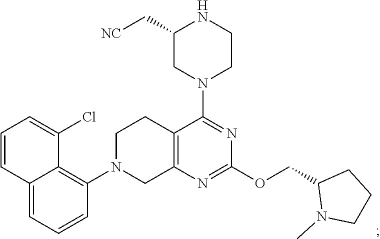

and
reacting

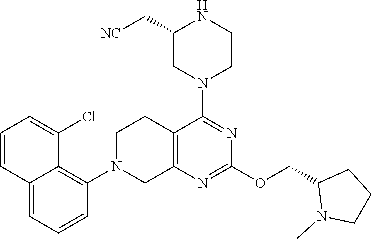

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting

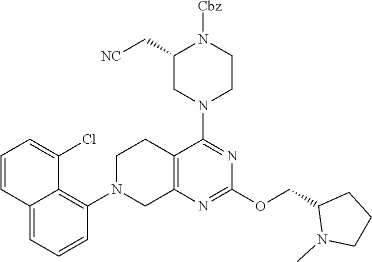

with p-toluenesulfonic acid in the presence of a first solvent and an anti-solvent to produce

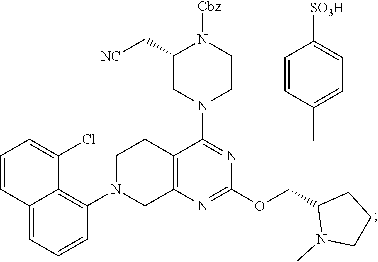

reacting

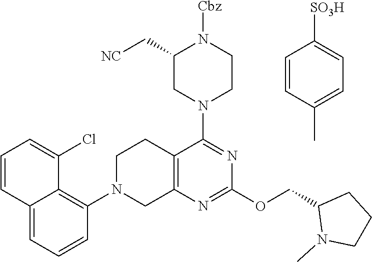

with a thiol or thiolate in the presence of a base and a polar aprotic solvent to produce

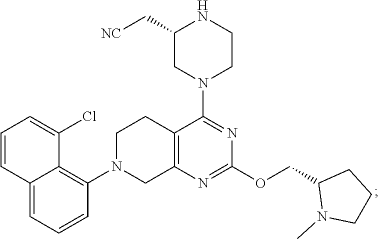

and
reacting

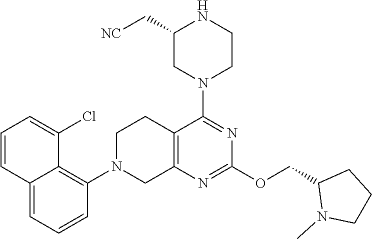

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting the free base of

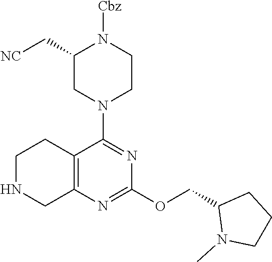

with

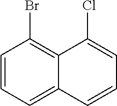 [SMILES: Clc1cccc2cccc(Br)c12]

in the presence of a palladium catalyst, a base, a phosphorous-based ligand, and an aprotic solvent to produce

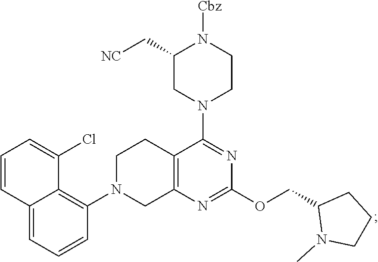

reacting

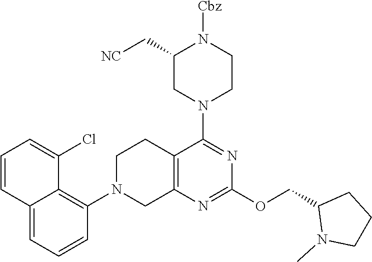

with p-toluenesulfonic acid in the presence of a first solvent and an anti-solvent to produce

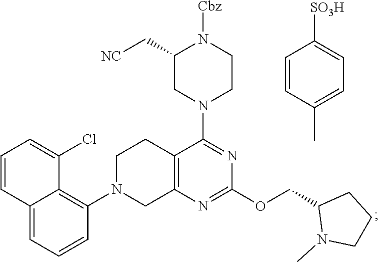

reacting

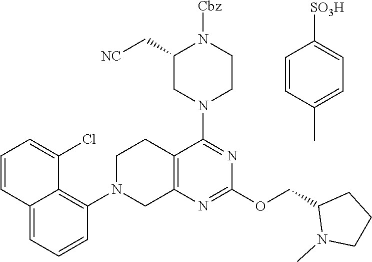

with a thiol or thiolate in the presence of a base and a polar aprotic solvent to produce

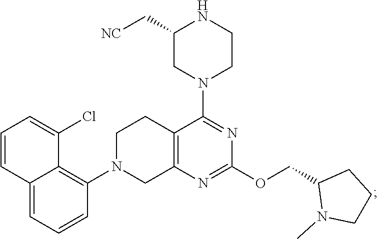

and
reacting

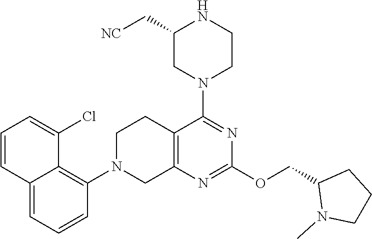

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting

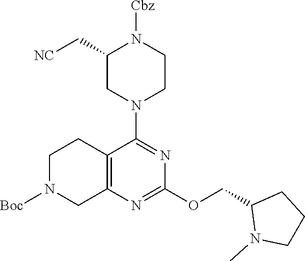

with an acid to remove a Boc protecting group to produce a salt or free base of

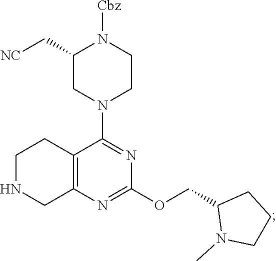

reacting the salt or free base of

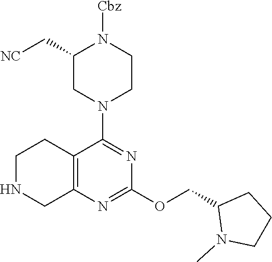

with

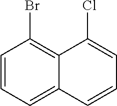 [SMILES: Clc1cccc2cccc(Br)c12]

in the presence of a palladium catalyst, a base, a phosphorous-based ligand, and an aprotic solvent to produce

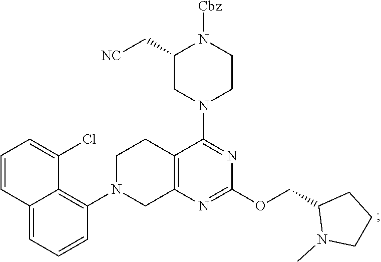

reacting

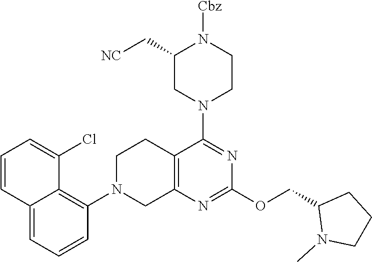

with p-toluenesulfonic acid in the presence of a first solvent and an anti-solvent to produce

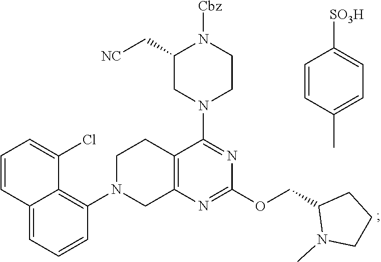

reacting

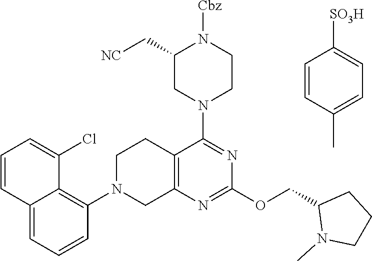

with a thiol or thiolate in the presence of a base and a polar aprotic solvent to produce

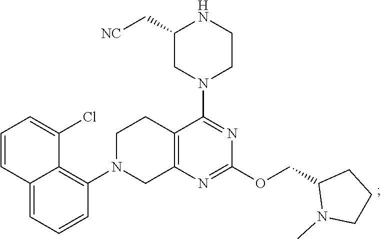

and
reacting

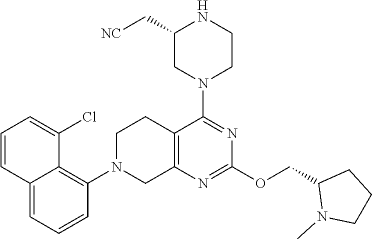

with 2-fluoroacrylic acid or alkali or metal salts of 2-fluoroacrylic acid with a coupling agent in the presence of a solvent and, optionally, a base to produce adagrasib.

In another aspect, the invention provides a method of synthesizing adagrasib, comprising the steps of:
reacting

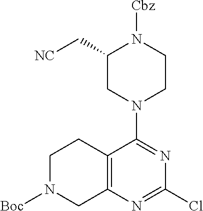 [SMILES: CC(C)(C)OC(=O)N1CCc2c(nc(Cl)nc2N2CCN(C(=O)OCc3ccccc3)[C@@H](CC#N)C2)C1]

with (S)-(1-methylpyrrolidin-2-yl)methanol in the presence of a palladium catalyst, a base, a phosphorous-based ligand, and an aprotic solvent to produce:

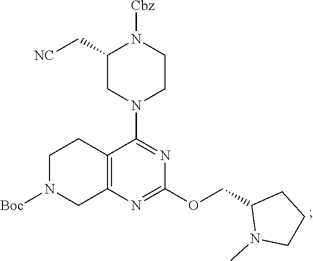

reacting

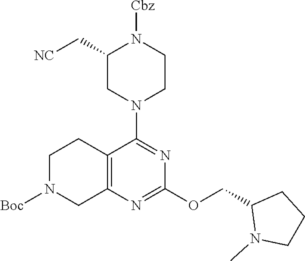

with an acid to remove a Boc protecting group to produce a salt or free base of:

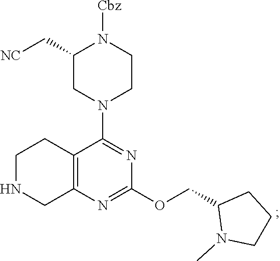

reacting the salt or free base of

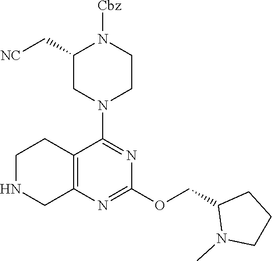

with

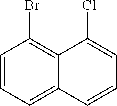 [SMILES: Clc1cccc2cccc(Br)c12]

in the presence of a palladium catalyst, a base, a phosphorous-based ligand, and an aprotic solvent to produce

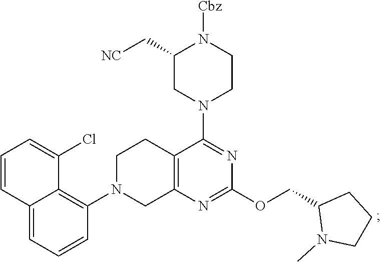

reacting

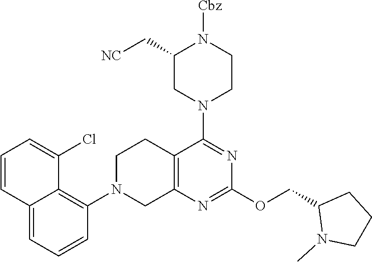

with p-toluenesulfonic acid in the presence of a first solvent and an anti-solvent to produce

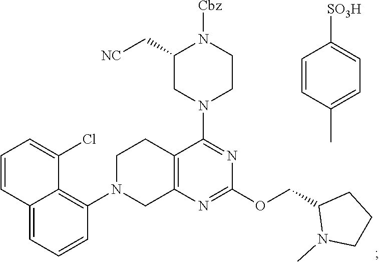

reacting

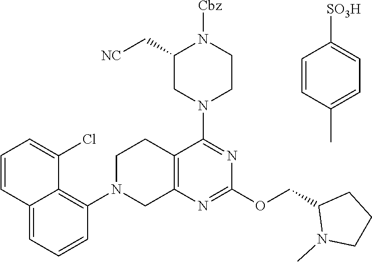

with a thiol or thiolate in the presence of a base and a polar aprotic solvent to produce

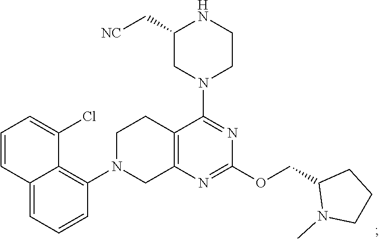

and
reacting

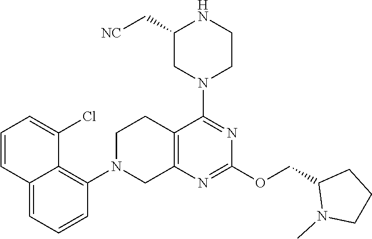

with 2-fluoroacrylic acid or alkali or metal salts of 2-fluoroacrylic acid with a coupling agent in the presence of a solvent and, optionally, a base to produce adagrasib.

In one aspect, the invention provides a method of synthesizing adagrasib, comprising the steps of:
a) reacting a compound of the following structure:

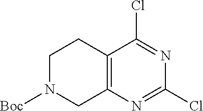 [SMILES: CC(C)(C)OC(=O)N1CCc2c(Cl)nc(Cl)nc2C1]

with a free base or a salt of a compound of the following structure:

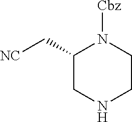 [SMILES: N#CC[C@H]1CNCCN1C(=O)OCc1ccccc1]

in the presence of a polar aprotic solvent and a base to produce a final compound of step (a) with the following structure:

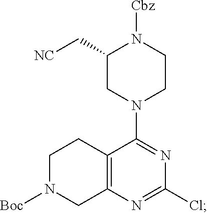 [SMILES: CC(C)(C)OC(=O)N1CCc2c(nc(Cl)nc2N2CCN(C(=O)OCc3ccccc3)[C@@H](CC#N)C2)C1]

b) reacting the final compound of step (a) with (S)-(1-methylpyrrolidin-2-yl)methanol in the presence of a palladium catalyst, a base, a phosphorous-based ligand, and an aprotic solvent to produce a final compound of step (b) with the following structure:

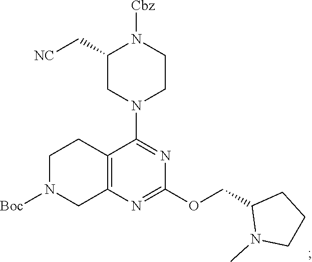

c) reacting the final compound of step (b) with an acid to remove a Boc protecting group from the final compound of step (b) to produce a salt or free base of a final compound of step (c) with the following structure:

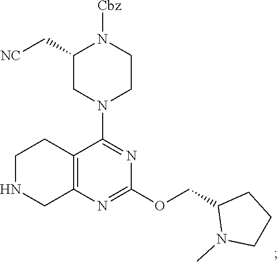

d) reacting the salt or free base of the final product of step (c) with

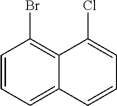 [SMILES: Clc1cccc2cccc(Br)c12]

in the presence of a palladium catalyst, a base, a phosphorous-based ligand, and an aprotic solvent to produce a final compound of step (d) with the following structure:

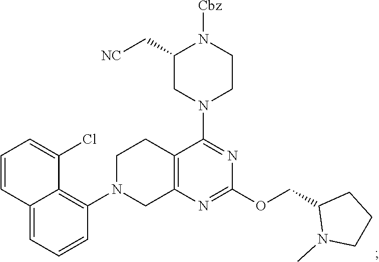

e) reacting the final compound of step (d) with p-toluenesulfonic acid in the presence of a first solvent and an anti-solvent to produce a final compound of step (e) with the following structure:

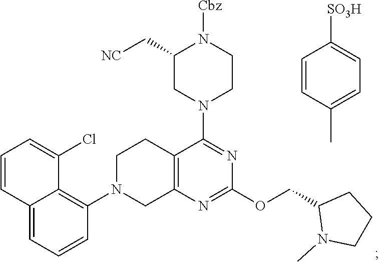

f) reacting the final compound of step (e) with a thiol or thiolate in the presence of a base and a polar aprotic solvent to produce a final compound of step (f) with the following structure:

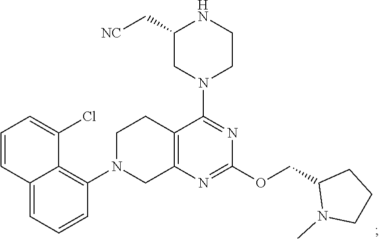

and
g) reacting the final compound of step (f) with 2-fluoroacrylic acid (or corresponding alkali or metal salts) and a coupling agent in the presence of a solvent and, optionally, a base to produce adagrasib.

In one aspect, the invention provides a method of synthesizing adagrasib, comprising the steps of:
a′) reacting a compound of the following structure:

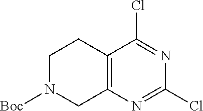 [SMILES: CC(C)(C)OC(=O)N1CCc2c(Cl)nc(Cl)nc2C1]

with the compound of the following structure:

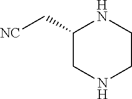

in the presence of a polar aprotic solvent and a base to produce the compound of the following structure:

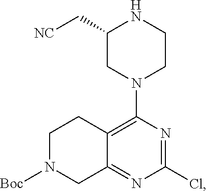 [SMILES: CC(C)(C)OC(=O)N1CCc2c(nc(Cl)nc2N2CCN[C@@H](CC#N)C2)C1]

and then
reacting this compound with benzyl chloroformate in the presence of sodium carbonate, methyl tert-butyl ether (MTBE) and water to produce a final compound of step (a′) with the following structure:

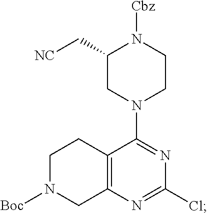 [SMILES: CC(C)(C)OC(=O)N1CCc2c(nc(Cl)nc2N2CCN(C(=O)OCc3ccccc3)[C@@H](CC#N)C2)C1]

reacting the final compound of step (a′) with (S)-(1-methylpyrrolidin-2-yl)methanol in the presence of a palladium catalyst, a base, a phosphorous-based ligand, and an aprotic solvent to produce a final compound of step (b) with the following structure:

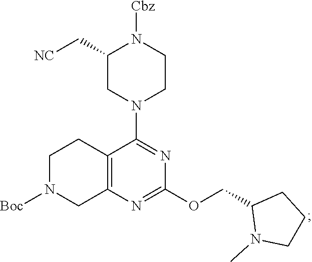

reacting the final compound of step (b) with an acid to remove a Boc protecting group from the final compound of step (b) to produce a salt or free base of a final compound of step (c) with the following structure:

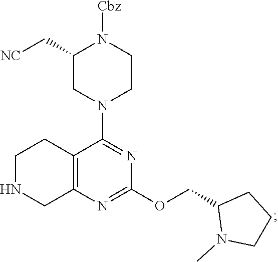

reacting the salt or free base of the final product of step (c) with

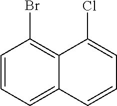 [SMILES: Clc1cccc2cccc(Br)c12]

in the presence of a palladium catalyst, a base, a phosphorous-based ligand, and an aprotic solvent to produce a final compound of step (d) with the following structure:

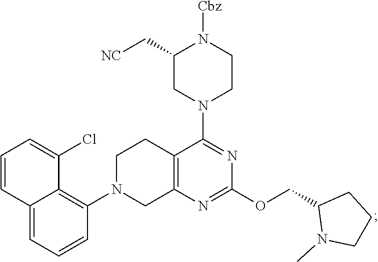

reacting the final compound of step (d) with p-toluenesulfonic acid in the presence of a first solvent and an anti-solvent to produce a final compound of step (e) with the following structure:

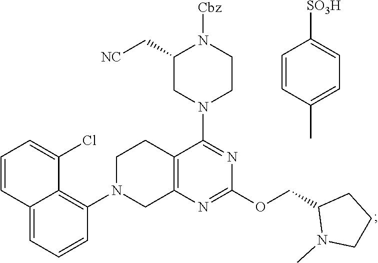

reacting the final compound of step (e) with a thiol or thiolate in the presence of a base and a polar aprotic solvent to produce a final compound of step (f) with the following structure:

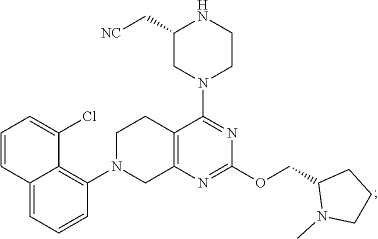

and
reacting the final compound of step (f) with 2-fluoroacrylic acid (or corresponding alkali or metal salts) and a coupling agent in the presence of a solvent and, optionally, a base to produce adagrasib.

In one embodiment, in step (a) and/or step (a′), the polar aprotic solvent is selected from the group consisting of dimethylacetamide (DMAc), dimethylformamide (DMF), 1,4-dioxane, tetrahydrofuran (THF), 2-methyltetrahydrofuran (2-MeTHF), acetonitrile (MeCN), dimethyl sulfoxide (DMSO), and N-methylpyrrolidone (NMP).

In one embodiment, in step (a) and/or step (a′), the polar aprotic solvent comprises, but is not limited to, one or more of the following: dimethylacetamide (DMAc), dimethylformamide (DMF), 1,4-dioxane, tetrahydrofuran (THF), 2-methyltetrahydrofuran (2-MeTHF), acetonitrile (MeCN), dimethyl sulfoxide (DMSO), and N-methylpyrrolidone (NMP).

In one embodiment, in step (a) and/or step (a′), the polar aprotic solvent is dimethylacetamide (DMAc).

In one embodiment, in step (a) and/or step (a′), the base is an organic base.

In one embodiment, the organic base is selected from the group consisting of N,N-diisopropylethylamine (DIPEA), triethylamine (Et₃N), 1,4-diazabicyclo[2.2.2]octane (DABCO), and 1,8-diazabicyclo[5.4.0]undec-7-ene (DBU).

In one embodiment, the organic base catalyst is N,N-diisopropylethylamine (DIPEA).

In one embodiment, the inorganic base catalyst is selected from the group consisting of carbonate, bicarbonate, and phosphate.

In step (a) and/or step (a′) above, the compound can be a salt of any organic or mineral acid, i.e. it can be any organic or mineral salt.

In one embodiment, the organic salt is selected from the group consisting of fumarate, tartrate, malate, and citrate.

In one embodiment, the organic salt is a fumarate salt.

In one embodiment, the mineral salt is selected from the group consisting of hydrochloride, hydrobromide, sulfate and phosphate.

In one embodiment, step (a) is carried out at a temperature from about −10° C. to about 80° C.

In one embodiment, step (a′) is carried out at a temperature from about 0° C. to about 10° C.

In one embodiment, in step (b), the palladium catalyst is in the oxidation state 0 or II.

In one embodiment, in step (b), the palladium catalyst is selected from the group consisting of Pd₂(dba)₃, Pd(dba)₂, and Pd(OAc)₂.

In one embodiment, in step (b), the palladium catalyst is pre-activated. Any pre-activated palladium catalyst can be used, including but not limited to the Buchwald series of Pd-G1 to Pd-G6 catalysts; the Organ series such as the PEPPSI (pyridine-enhanced precatalyst preparation stabilization and initiation), etc.

In one embodiment, in step (b), the base is an organic base.

In one embodiment, the organic base is selected from the group consisting of DIPEA, Et₃N, DABCO, and DBU.

In one embodiment, in step (b), the base is an inorganic base.

In one embodiment, the inorganic base is selected from the group consisting of carbonate, bicarbonate, and phosphate.

In one embodiment, in step (b), the phosphorous-based ligand is selected from the group consisting of a monodentate phosphorous-based ligand and a bidentate phosphorous-based ligand.

Monodentate phosphorous-based ligands may have the general formula PR₃ (wherein R can be Cy, tBu, Ph, or various combinations thereof, etc).

Bidentate phosphorous-based ligands include but are not limited to (2,2′-bis(diphenylphosphino)-1,1′-binaphthyl) (R), (S) and rac-(BINAP), XantPhos, Bis[(2-diphenylphosphino)phenyl] ether (DPEPhos), ferrocene-based backbone ligands (e.g., 1,1′-bis(diphenylphosphino)ferrocene (dppf)), and others.

In one embodiment, in step (b), the aprotic solvent is selected from the group consisting of toluene, 1,4-dioxane, THF, 2-MeTHF, MeCN, DMSO, and NMP.

In one embodiment, step (b) is carried out at a temperature from about 20° C. to about 120° C.

In step (c), removal of Boc protecting group can be done using any mineral acid, including but not limited to hydrochloric acid, sulfuric acid, nitric acid and phosphoric acid. Any organic acid can be used, including but not limited to trifluoroacetic acid (TFA), citric acid, tartaric acid, etc.

In one embodiment, in step (c), the final compound can be a tartrate salt or a citrate salt.

In one embodiment, in step (d), the palladium catalyst is in the oxidation state 0 or II.

In one embodiment, in step (d), the palladium catalyst is selected from the group consisting of Pd₂(dba)₃, Pd(dba)₂, and Pd(OAc)₂.

In one embodiment, in step (d), the palladium catalyst is pre-activated. Any pre-activated palladium catalyst can be used, including but not limited to the Buchwald series of Pd-G1 to Pd-G6 catalysts; the Organ series such as the PEPPSI, etc.

In one embodiment, in step (d), the base catalyst is an organic base catalyst.

In one embodiment, the organic base is selected from the group consisting of DIPEA, Et₃N, DABCO, and DBU.

In one embodiment, in step (d), the base is an inorganic base.

In one embodiment, the inorganic base is selected from the group consisting of carbonate, bicarbonate, and phosphate.

In one embodiment, in step (d), the phosphorous-based ligand is selected from the group consisting of a monodentate phosphorous-based ligand and a bidentate phosphorous-based ligand.

Monodentate phosphorous-based ligands may have the general formula PR₃ (wherein R can be Cy, tBu, Ph, etc).

Bidentate phosphorous-based ligands include but are not limited to BINAP, XantPhos, DPEPhos, ferrocene-based backbone ligands (e.g., dppf), and others.

In one embodiment, in step (d), the aprotic solvent is selected from the group consisting of toluene, 1,4-dioxane, THF, 2-MeTHF, MeCN, DMSO, and NMP.

In one embodiment, step (d) is carried out at a temperature from about 20° C. to about 120° C.

In one embodiment, in step (e), the first solvent is selected from the group consisting of a ketone containing solvent and acetonitrile. In one embodiment, the ketone containing solvent is selected from the group consisting of acetone, methyl isobutyl ketone (MIBK), and methyl ethyl ketone (MEK).

In one embodiment, in step (e), the anti-solvent is selected from the group consisting of 2-MeTHF and isopropyl acetate (IPAc).

In one embodiment, step (e) is carried out at a temperature from about 20° C. to about 120° C.

In one embodiment, in step (f), the thiol or thiolate is selected from the group consisting of 2-mercaptoethanol, dithiothreitol (DTT), 2-(dimethylamino)ethanethiol hydrochloride, and R—SY, wherein R is selected from the group consisting of H, alkyl, and aryl, and wherein Y is selected from the group consisting of H, alkali and metal salts.

In one embodiment, in step (f), the base is an organic base.

In one embodiment, the organic base is selected from the group consisting of DIPEA, Et₃N, DABCO, and DBU.

In one embodiment, wherein in step (f), the base is an inorganic base.

In one embodiment, the inorganic base is selected from the group consisting of carbonate, bicarbonate, and phosphate.

In one embodiment, in step (f), the polar aprotic solvent is selected from the group consisting of DMAc, DMF, 1,4-dioxane, THF, 2-MeTHF, MeCN, DMSO, and NMP.

In one embodiment, step (f) is carried out at a temperature from about 20° C. to about 120° C.

In one embodiment, in step (g), the solvent is selected from the group consisting of DMAc, DMF, 1,4-dioxane, THF, 2-MeTHF, MeCN, DMSO, dichloromethane (DCM), ethyl acetate (EtOAc), IPAc, and NMP.

In one embodiment, in step (g), 2-fluoroacrylic acid can be used in the neutral form, free acid, or ionic form (as a metal or alkali salt).

In one embodiment, in step (g), the coupling agent is selected from the group consisting of propylphosphonic anhydride (T3P®), carbonyldiimidazole (CDI), the carbodiimide (e.g. dicyclohexylcarbodiimide (DCC), diisopropylcarbodiimide (DIC), ethyl-(N′,N′-dimethylamino)propylcarbodiimide hydrochloride (EDC·HCl)), the phosphonium ((benzotriazol-1-yloxy)tris(dimethylamino)phosphonium hexafluorophosphate (BOP), (benzotriazol-1-yloxy)tripyrrolidinophosphonium hexafluorophosphate (PyBOP)) and uronium (O-(benzotriazol-1-yl)-N,N,N′,N′-tetramethyluronium hexafluorophosphate (HBTU), O-(7-azabenzotriazol-1-yl)-N,N,N′,N′-tetramethyluronium hexafluorophosphate (HATU)).

In one embodiment, in step (g), the base is an organic base.

In one embodiment, the organic base is selected from the group consisting of DIPEA, Et₃N, DABCO, and DBU.

In one embodiment, wherein in step (g), the base is an inorganic base.

In one embodiment, the inorganic base is selected from the group consisting of carbonate, bicarbonate, and phosphate.

In one embodiment, step (g) is carried out at a temperature from about −10° C. to about 50° C.

The following Examples are intended to illustrate further certain embodiments of the invention and are not intended to limit the scope of the invention.

### Example 1

### Step (a)

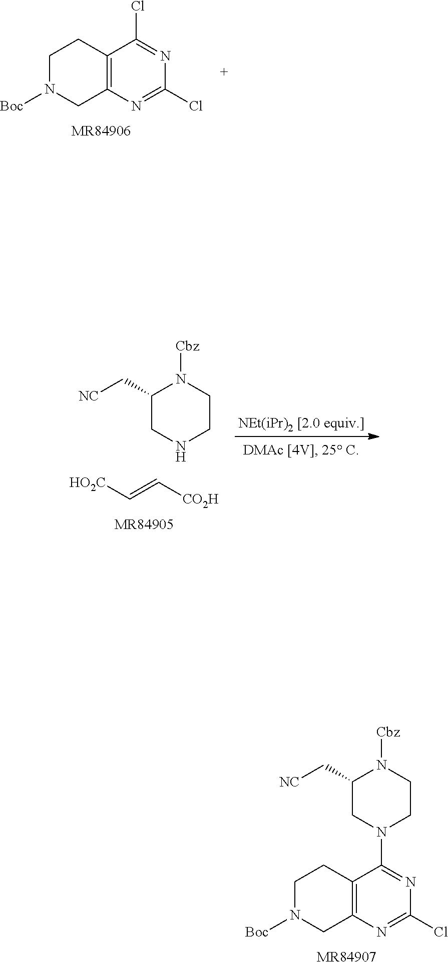

N,N-Dimethylacetamide (371.9 kg) was charged into a 3000 L glass-lined reactor and stirred for 5-10 min. The mixture was sampled to confirm moisture level below 0.1%, and Karl-Fischer analysis demonstrated that the actual water quantity was 0.01%. The MR84905 Fumarate (ASYM-124583) (136.7 kg, 135.2 kg corrected for purity, 360.2 mol, 1.1 equiv.) was added into the mixture at a temperature between 10-30° C., and the mixture was stirred for 15-30 min. The mixture was adjusted to a temperature between 10-20° C. and set to 13.6° C. Diisopropylethylamine (DIPEA, 88.4 kg, 683.9 mol, 2.0 equiv.) was added to the reaction mixture, followed by addition of MR84906 (ASYM-124584) (106.3 kg, 104.0 kg corrected by HPLC assay, 341.9 mol, 1.0 equiv.). The reactor walls were rinsed with N,N-dimethylacetamide (41.7 kg), and the mixture was stirred for 30 to 60 min.

Following this, the reaction mixture was sparged with nitrogen from the lower port for 5-10 min. The mixture was allowed to react at 10-20° C., and after 2 h, the mixture was sampled by HPLC every 1-3 h until the area % of MR84906 was ≤1.0 area %. After 4 h and 26 min stirring at 15° C., the area % of MR84906 was 0.3%. At this time, the reaction was diluted with MTBE (754.1 kg) at 10-30° C. The temperature of the reaction mixture was adjusted to be between 30-40° C. and set at 32.6° C. Purified water (532.9 kg) was added into the mixture at a temperature of 30-40° C. and stirred for 20-30 min. Stirring was stopped and the layers were allowed to settle to form clearly defined phases. The aqueous layer was removed at 30-40° C. The organic phase was then washed with a sodium chloride solution at 30-40° C., which was prepared from purified water (528.8 kg) and sodium chloride (27.6 kg). The biphasic mixture was stirred for an additional 20-30 min, settled to form distinct layers, and then the aqueous phase was removed at 30-40° C. The organic phase remaining in the reactor was concentrated at a temperature ≤40° C. under reduced pressure (≤−0.06 MPa) until 780.0-884.0 L (7.5˜8.5 vol) remained. The temperature was adjusted to be between 30-40° C. and set to 30.9° C. The mixture was stirred for 4-6 h at a temperature between 30-40° C. at which point a substantial amount of solids had precipitated from the mixture. n-Heptane (143.6 kg) was added into the mixture at 30-40° C. which resulted in a thick slurry. The slurry was slowly cooled to a temperature between 15-25° C. and set at 23.5° C. Temperature was maintained at this level during the crystallization for 2-3 h.

The supernatant was sampled for assay analysis of MR84907. An assay of ≤0.5% was desired and the actual value was 0.4%. The slurry was transferred and filtered with an agitated Nutsche Filter Dryer. The reactor was rinsed with n-heptane (71.0 kg) which was then transferred into the filter to rinse the filter cake. The filter cake was swept with nitrogen for 1-2 h, dried at T_{jacket}≤45° C. with stirring, until the sum of MTBE and n-heptane residuals were less than 2.0% and the water level was less than 1.0% as demonstrated by KF analysis. Actual values were 0.0% and 0.3%, respectively. After drying was completed, the filter jacket was cooled to a temperature between 20-30° C. (22.4° C., actual). MR84907 was obtained as an off-white solid (160.4 kg, 160.4 kg corrected for purity, 100.5 assay w/w %, 89.0% yield).

M.p.: 128.7-128.8° C.

¹H NMR (400 MHz, DMSO-d₆) δ ppm 1.44 (s, 9H), 2.62-2.75 (m, 2H), 2.92 (br, J=5.6 Hz, 1H), 2.95-3.12 (m, 2H), 3.17-3.34 (m, 3H), 3.61-3.70 (m, 1H), 3.85-4.03 (m, 3H), 4.24-4.38 (m, 1H), 4.40-4.51 (m, 1H), 4.54-4.63 (m, 1H), 5.14 (s, 2H), 7.28-7.47 (m, 5H).

¹³C NMR (101 MHz, DMSO-d₆) δ ppm 18.2, 25.5, 26.8, 28.0, 47.1, 47.9, 48.1, 48.3, 48.7, 66.8, 79.5, 113.9, 118.3, 127.6, 127.9, 128.4, 136.4, 153.5, 154.2, 156.2, 163.5, 165.6.

HRMS (ESI) calculated for C₂₆H₃₂ClN₆O₄: 527.2174 [M+H]⁺. Found: 527.2283.

Alternatively, step (a′) can be utilized to obtain MR84907.

### Step (a′)

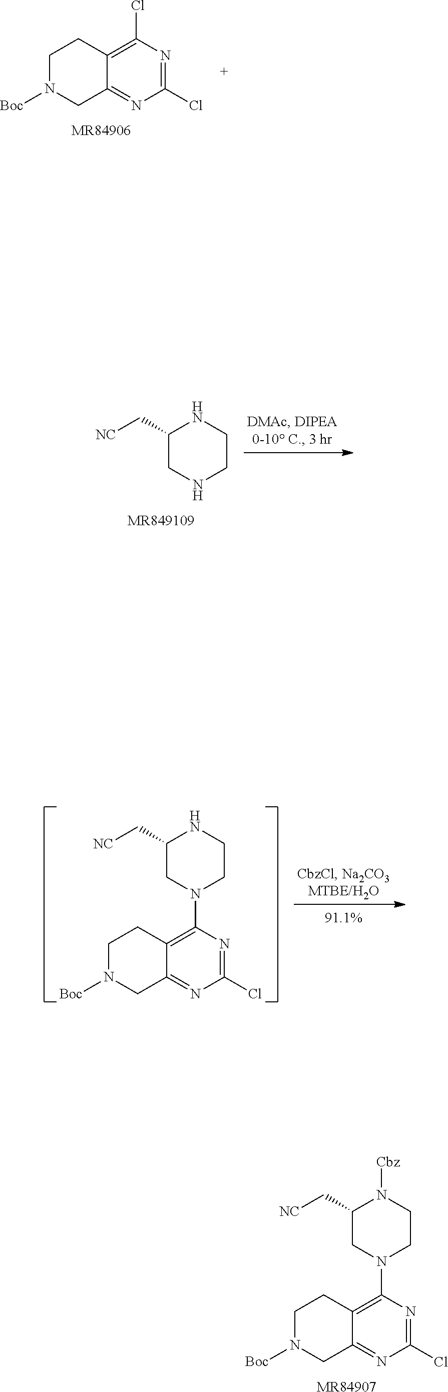

DMAc (120 mL) and MR849109 (30 g, 0.153 mol, 1 equiv.) were charged into a 1000 mL reactor at 20° C. Diisopropylethyl amine (79.2 g, 0.612 mol, 4 equiv.) was added dropwise, maintaining a temperature between 10 to 20° C., and the mixture was cooled to 0 to 10° C. MR84906 (46.5 g, 0.153 mol, 1 equiv.) was charged in portions keeping the temperature between 0 to 10° C. The reaction mixture was held at 0 to 10° C. for 1-2 hr prior to sampling for analysis of residual MR84906. Once the sample reached ≤2.0% of MR84906, MTBE (150 mL) and sodium carbonate (8.11 g, 0.0765 mol, 0.5 equiv. in 90 mL of water) were added to the reaction mixture at 0 to 10° C. If the reaction was unable to meet the 2.0% IPC for MR84906, additional MR849109 was added based on the quantity of residual MR84906. CbzCl (33.9 g, 0.199 mol, 1.3 equiv.) was added to the biphasic reaction dropwise at 0 to 10° C. The mixture was then warmed to 20-30° C. and held 2 hours. The mixture was checked for residual intermediate to be less than 1.0%, and additional CbzCl was added correspondingly based on the quantity of residual intermediate. Once the level of intermediate was ≤1.0%, MTBE (150 mL) and water (60 mL) were added at 20 to 40° C. and the mixture was stirred for 30 min to give a homogeneous mixture. The biphasic mixture was separated at 30-40° C., and the aqueous layer was discarded. The organic layer was washed with 10% aq. NaCl (150 mL). The organic layer was concentrated to 8 volumes (240 mL) at a temperature below 45° C. 0.3 wt % of MR84907 seed crystals were added to promote crystal growth at 30-40° C. The mixture was stirred for 4 to 6 hr, and significant crystal growth was observed. N-Heptanes (60 mL) were charged into the mixture at 30 to 40° C. Stirring continued for 1-2 hr. The mixture was slowly cooled to 25-35° C. at a rate of 3 to 5° C./hr, and then stirred for 10 hr. The slurry was filtered and the solids were rinsed with 30 mL of MTBE/n-Heptane (4:1). The solids were dried at no more than 45° C. until the sum of MTBE and n-Heptane were no more than 2.0% and the water content was less than 1.0%. 74.0 g of MR84907 was obtained, having purity of 99.2% as judged by HPLC area percent, and 98.31 assay wt %, resulting in an isolated yield of 91.1%. Spectroscopic and chromatographic characterization matched the reported data of MR84907.

### Example 2

### Steps (b) and (c)

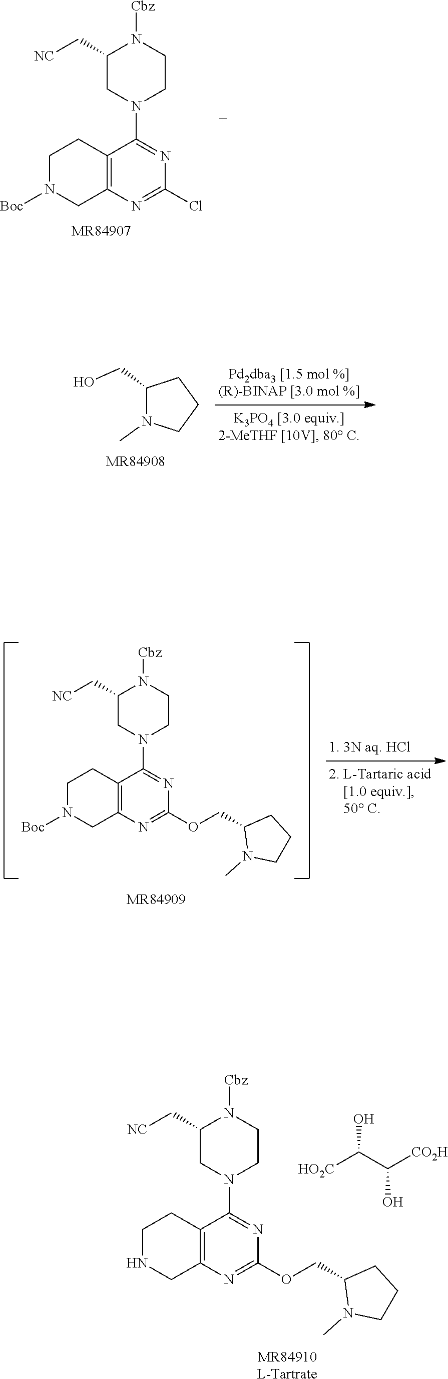

2-MeTHF (1336.9 kg) was charged into a 5000 L glass-lined reactor. After 5-10 min of stirring, the moisture content was checked by Karl-Fischer analysis to confirm a level below 0.5% (0.03%, actual). MR84907 (156.5 kg, 156.5 kg corrected by HPLC assay, 297.0 mol, 1.0 equiv.) was added into the mixture at a temperature between 10-30° C. and was stirred until solids were completely dissolved as confirmed by a visual check. Anhydrous potassium phosphate (190.0 kg, 891.0 mol, 3 equiv.) was added into the mixture at a temperature between 10-30° C., followed by addition of (S)-(1-methylpyrrolidin-2-yl)methanol (MR84908, 51.1 kg, 443.7 mol, 1.5 equiv.) at a temperature between 10-30° C. The mixture was degassed by bubbling nitrogen from the lower port of reactor at 10-30° C. until the oxygen content reached a level ≤0.1% (0.01% actual oxygen content). (R)-BINAP (5.6 kg, 8.9 mol, 3 mol %) and Pd₂(dba)₃ (4.1 kg, 4.5 mmol, 1.5 mol %) were added into the mixture at a temperature between 10-30° C. under the protection of nitrogen. After the addition was completed, the mixture was degassed for 0.5-1 h by bubbling nitrogen from the lower port until the oxygen content reached a level 0.1% (0.01% actual oxygen content). The mixture was then heated to 70-80° C. (75.9° C. actual). The mixture was allowed to react at 70-80° C. for 20 h at which point the mixture was sampled for HPLC purity analysis every 4-8 h until the area % of MR84907/(MR84907+MR84909) was ≤3.0%. After 30 h and 8 min, the value was 0.6%. The mixture was then cooled to a temperature between 10-30° C. (22.9° C. actual), at which point purified water (780.0 kg) was added into mixture at a temperature between 15-30° C. It should be noted that the water addition is highly exothermic during the first 10% of the addition and that the addition rate should be carefully controlled. Following the addition of water, the mixture was stirred for 20-30 min at a temperature of 15-30° C., and the layers were allowed to settle before separation. The bottom aqueous layer was removed from the bottom valve, and then the organic phase was washed with a sodium chloride solution which was prepared by addition of sodium chloride (17.2 kg) to purified water (305.6 kg). The mixture was stirred for 20-30 min at a temperature of 15-30° C., and the layers were allowed to settle before separation. The bottom aqueous layer was removed from the bottom valve. A hydrochloric acid solution was added to the organic phase remaining in the reactor at 15-25° C. The hydrochloric acid solution was prepared by addition of concentrated hydrochloric acid (210.8 kg) to purified water (690.2 kg). The mixture was stirred for 20-30 min at a temperature between 15-25° C., and the layers were allowed to settle before separation. The lower aqueous phase contained the product and was transferred into a 5000 L reactor to be telescoped with Step 3 (Boc-deprotection and crystallization as the L-Tartaric acid salt). The aqueous phase was allowed to react at a temperature between 15-25° C. (21.1-22.5° C., actual temperature).

After 15 h, the mixture was sampled for HPLC purity analysis every 1-3 h until the area % of MR84909/(MR84909+MR84910) was ≤1.0% (0.1 area % after 22 h and 57 min). 2-MeTHF (269.3 kg) was added to the reaction mixture at 15-25° C. The mixture was stirred for another 20-30 min at 15-25° C. and then the layers were allowed to settle before separating them and discarding the organic layer (2-MeTHF). The pH of the aqueous phase was adjusted to 8-9 at a temperature between 15-25° C. with a potassium carbonate solution (957.6 kg) which was prepared from purified water (667.8 kg) and potassium carbonate (289.3 kg). After a pH between 8-9 was reached, the mixture was continued to stir for another 0.5-1 h, and the pH was retested until it did not change (pH=8, actual value). The mixture was adjusted to a temperature between 25-35° C. Then sodium chloride (249.8 kg) was added into the mixture. The mixture was stirred until the solid was dissolved completely as confirmed by a visual check. 2-MeTHF (1338.6 kg) was added to the mixture at a temperature between 15-25° C. The mixture was stirred for 20-30 min and then the layers were allowed to settle. The organic phase was collected, and the aqueous phase was returned to the reactor for a second extraction with 2-MeTHF (666.9 kg). The two 2-MeTHF layers were combined, transferred to a 5000 L glass-lined reactor, and concentrated at a temperature ≤40° C., under reduced pressure (P≤−0.06 MPa) until 546˜702 L (3.5˜4.5 vol) remained. 2-MeTHF (534.0 kg) was added into the mixture at a temperature ≤40° C. The mixture was sampled for moisture content to confirm a value ≤0.5%. Karl-Fischer analysis showed that the moisture content was 0.6%. As a result, the mixture was again concentrated at a temperature ≤40° C., under reduced pressure (P≤−0.06 MPa) until 546˜702 L (3.5˜4.5 vol) remained. 2-MeTHF (523.9 kg) was added into the mixture at a temperature ≤40° C. The mixture was sampled for moisture content to confirm a value ≤0.5%. Karl-Fischer analysis showed that the moisture content was 0.2%. The mixture was then circulated through a CUNO filtration system at a temperature between 25-40° C. The pipes and CUNO filter were rinsed with 2-MeTHF (201.3 kg). The mixture was heated to a temperature between 45-55° C. (50.0° C. actual). An L-tartaric acid solution, which was prepared by dissolving L-tartaric acid (43.8 kg, 291.2 mol, 1.0 equiv.) in isopropanol (499.3 kg) at a temperature between 45-55° C. was added into the reaction mixture at a temperature between 45-55° C. The mixture was stirred at a temperature between 45-55° C. for 1-2 h. The mixture was cooled slowly to a temperature between 20-30° C. (28.6° C. actual) to trigger crystallization.

After 2 h, the mixture was sampled for assay analysis of MR84910 in the supernatant layer every 1-3 h until it was ≤0.5 wt % as judged by HPLC. In the first sample, the wt % of MR84910 was 0.3%, and so the mixture was filtered with a stainless steel Nutsche centrifuge. The reactor walls were rinsed with 2-MeTHF (134.2 kg), and then transferred into the filter to rinse and filter the filter cake. The wet filter cake was dried in a rotary conical dryer at Tjacket≤40° C., P≤−0.06 MPa until the sum of 2-MeTHF and isopropanol residuals were ≤2.0% as judged by GC (1.6% actual). After the drying was completed, the jacket was cooled to a temperature between 20-30° C., and the intermediate (MR84910) was collected in plastic bags, filled with nitrogen, put into aluminum bags and sealed, and then stored under dry conditions. The MR84910 L-tartrate was very sensitive to moisture and should be handled under nitrogen protection. The product was obtained as a white solid (167.8 kg, 156.2 kg corrected for assay purity, 71.8% assay w/w % (free base), 80.2% yield).

M.p.: 61.2-61.3° C.

¹H NMR (500 MHz, D₂O) δ ppm 1.90-2.00 (m, 1H), 2.02-2.10 (m, 1H), 2.11-2.20 (m, 1H), 2.26-2.37 (m, 1H), 2.71-2.82 (m, 1H), 2.82-2.96 (m, 4H), 2.99 (br s, 3H), 3.07-3.22 (m, 2H), 3.23-3.32 (m, 2H), 3.33-3.40 (m, 1H), 3.46 (s, 1H), 3.64-3.84 (m, 2H), 3.98 (br dd, J=12.32, 6.30 Hz, 3H), 4.22 (s, 2H) 4.30 (s, 2H), 4.45-4.53 (m, 1H), 4.62 (br dd, J=12.59, 2.74 Hz, 2H), 5.09 (d, J=7.67 Hz, 2H), 7.24-7.38 (m, 5H).

¹³C NMR (126 MHz, D₂O) δ ppm 19.3, 22.7, 26.9, 39.7, 41.6, 41.9, 45.9, 47.7, 48.7, 49.3, 57.9, 59.4, 65.1, 68.6, 70.5, 74.2, 108.0, 119.6, 128.5, 129.1, 129.4, 136.7, 156.9, 158.4, 162.2, 166.7, 178.4.

HRMS (ESI) calculated for C₂₇H₃₆N₇O₃: 506.2880 [M+H]⁺. Found: 506.3000.

Alternatively, steps (b′) and (c′) can be utilized to obtain MR84907. These steps allow to decrease the amount of palladium consumed in production of MRTX849. This technology can reduce the palladium loading by three- to six-fold. The key difference is the use of Xantphos as a ligand instead of R-BINAP, and the grinding of K₃PO₄ to reduce the phosphate's particle size.

### Steps (b′) and (c′)

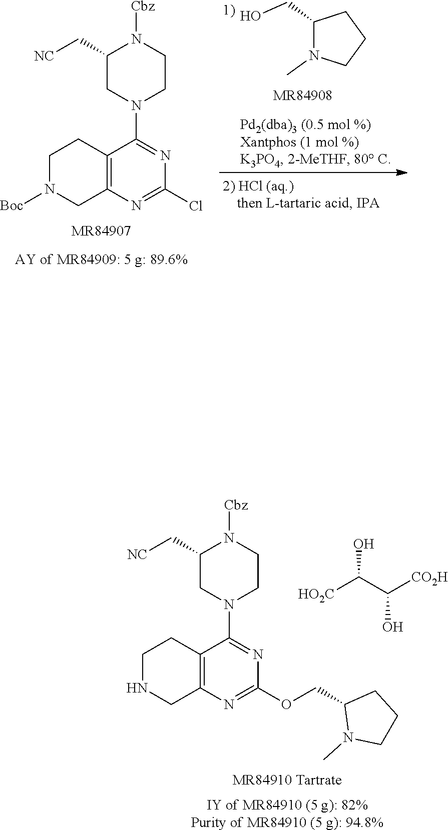

MR84908 (1.64 g, 14.23 mmol, 1.50 equiv.), anhydrous K₃PO₄ (6.04 g, 28.46 mmol, 3.00 equiv.), XantPhos (43.9 mg, 75.9 μmol, 0.008 equiv.), Pd2(dba)3 (43.4 mg, 47.4 μmol, 0.005 equiv.), MR84907 (5.0 g, 9.49 mmol, 1.00 equiv.) and dry 2-MeTHF (30 mL, 6.0 V) were charged into a reactor equipped with an overhead stirrer under nitrogen atmosphere. A rotor-stator homogenizer was added to the reaction manifold to mill the potassium phosphate. The overhead stirrer was turned on and set to an agitation rate of 300 rpm. The mixture was purged with nitrogen for 5 min. The homogenizer was engaged and set to a rate 15,000 rpm. The mixture was heated to 75° C. After 5 hr, the homogenizer was turned off and the reaction mixture was sampled for HPLC analysis to monitor completion of the reaction. Once the level of MR84907 was ≤2.0%, the mixture was cooled to 20° C. If the level of MR84907 was more than 2.0% after 20 hr, additional catalyst was added to commensurate with the amount of residual MR84907. Water (25 mL, 5V) was charged into the reaction mixture while maintaining a temperature below 25° C., and the mixture was stirred until all solids were completely dissolved. The biphasic layer was separated, collecting the organic layer. Fresh MeTHF (25 mL, 5V) was added to the aqueous layer and stirred for 15 min. The phases were split and the organic layer was retained. The two organic layers were combined and washed with 5 wt % aq. NaCl solution (2V). The biphasic layer was separated and the aqueous layer was discarded. 14 wt % aq. HCl (5 equiv.) was added to the organic layer while maintaining a temperature of 15-25° C. The biphasic mixture was allowed to settle and the phases were split. The aqueous layer was retained, and stirred at 20° C. until HPLC analysis indicated that the level of MR84909 was ≤1.0%. MeTHF (10 mL, 2V) was charged to the agitated mixture. The phases were allowed to settle and then they were separated, collecting the aqueous layer. 50 wt % aq. K₂CO₃ (7 equiv.) was added to the aqueous layer at 15-25° C. until the pH reached a value of 9-10. Solid NaCl (5.0 g, 1.0 w/w) was added to the aqueous mixture, and the mixture was stirred until all solids dissolved. The mixture was extracted with MeTHF (50 mL, 10V). The phases were split and the organic layer was retained. The aqueous portion was back-extracted with MeTHF (25 mL, 5V). The layers were split and the organic fractions were combined and concentrated at 40° C. under reduced pressure. MeTHF (20 mL, 4V) was added to the concentrates, which were again concentrated at 40° C. under reduced pressure. MeTHF (50 mL, 10V) was added to the concentrates and the mixture was heated to 45-55° C. A solution of L-tartaric acid (1.42 g, 9.49 mmol, 1 equiv.) in isopropanol (20 mL, 4V) was added at 45-55° C. The mixture was agitated and held at this temperature for 2 h. The mixture was slowly cooled to 20° C. at a rate of 3-5° C./min. The mixture was held at 20° C. for 16 hr. The mixture was filtered, and the filter cake was rinsed with MeTHF (10 mL, 2V) three times under inert atmosphere. The cake was dried at ≤40° C. 5.39 g of MR84910 tartrate was obtained having an HPLC purity of 94.8%.

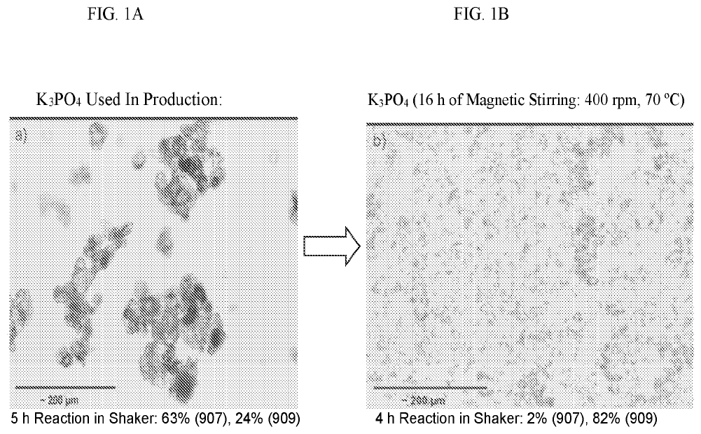

FIGS. 1A and 1B illustrate the advantages of steps (b′) and (c′) over steps (b) and (c). FIG. 1A depicts phosphate particle sizes when steps (b) and (c) are employed. FIG. 1B depicts phosphate particle sizes when steps (b′) and (c′) are employed. FIG. 1B shows reduction in phosphate particle size. The particle size appears to go from approximately 200 μm (FIG. 1A) down to approximately 20-50 μm (FIG. 1B) when milled or when magnetic stirring breaks apart the phosphate. This increases the rate of reaction as one can see in FIG. 1A and FIG. 1B. MR84909 increases from 24% to 82% AY yield under conditions which are otherwise identical.

### Example 3

### Steps (d) and (e)

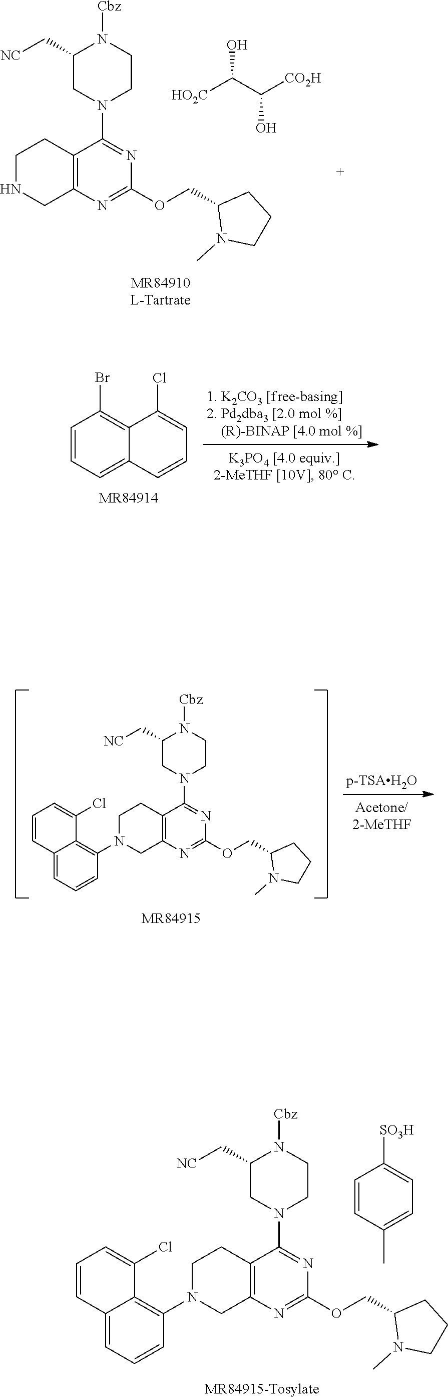

Purified water (467.7 kg) was charged into a 3000 L glass-lined reactor at 20-30° C. and stirring was started. MR84910 L-Tartrate (163.1 kg, 117.1 kg as MR84910 free base corrected by HPLC assay, 231.6 mol, 1.0 equiv.) was added into the reactor under stirring at a temperature between 20-30° C. The mixture was then stirred until solid was completely dissolved as confirmed by a visual check. 2-MeTHF (1218.1 kg) was then added into the mixture. The mixture was adjusted to a pH of 8-9 at a temperature between 10-30° C. with a 30% solution of potassium carbonate (319.6 kg) which was prepared with potassium carbonate (96.2 kg) and purified water (223.4 kg). The mixture then continued to stir for another 0.5 h and the solution pH was retested for confirmation (pH 8, actual). Sodium chloride (117.2 kg) was then added into the mixture, and the mixture was stirred for 20-30 min at a temperature between 10-30° C. The layers were allowed to settle before separation. The aqueous phase was discarded, and the top organic phase was washed with a sodium chloride solution which was prepared from sodium chloride (58.6 kg) and purified water (233.9 kg). The mixture was stirred for 20-30 min at a temperature between 10-30° C. and then the layers were allowed to settle before separation. The organic phase was concentrated at T_{jacket}≤50° C. under reduced pressure (P≤−0.08 MPa) until 3-4 vol (351-468 L) remained, and then 2-MeTHF (305.2 kg) was added into the mixture. The mixture was again concentrated at T_{jacket}≤50° C. under reduced pressure (P≤−0.08 MPa) until 3-4 vol (351-468 L) remained. The mixture was sampled for moisture content to confirm a value below ≤0.5%. Karl-Fischer analysis revealed a level of 1.3% moisture, and so the drying process was repeated. Another charge of 2-MeTHF (302.2 kg) was added into the mixture, which was again concentrated at T_{jacket}≤50° C. under reduced pressure (P≤−0.08 MPa) until 3-4 vol (351-468 L) remained. The mixture was sampled for moisture content to confirm a value below ≤0.5%, and this time the Karl-Fischer analysis revealed a moisture level of 0.2%. The mixture was adjusted to a temperature between 10-30° C. (27.1° C., actual), and the mixture was circulated through a CUNO filtration system. 2-MeTHF (497.4 kg) was added into a separate 5000 L glass-lined reactor, followed by addition of anhydrous potassium phosphate (196.8 kg, 926 mol, 4 equiv.) at 10-30° C. The mixture was stirred for 20-30 min after the addition and then was recycled for 2-3 h through a wet mill at 10-30° C. until the particle size D(90) was less than 50 μm (22 μm, actual). The MR84910 solution in the 3000 L reactor was transferred to the 5000 L reactor, passing through the CUNO filter, and then the 3000 L reactor was rinsed with 2-MeTHF (202.5 kg), transferred to the 5000 L reactor through the CUNO filter. MR84914 (58.5 kg, 243 mol, 1.05 equiv.) was added into the mixture in 5000 L reactor at a temperature between 10-30° C., and then the mixture was degassed by bubbling nitrogen from the lower port of reactor until oxygen content reached a level below 0.1% (0.00%, actual). (R)-BINAP (5.8 kg, 9.3 mol, 4 mol %) and Pd(OAc)₂ (1.0 kg, 4.6 mol, 2 mol %) were added into the mixture at 10-30° C. under nitrogen protection, and the mixture was again degassed by bubbling nitrogen from the lower port of reactor until oxygen content reached a level below 0.1% (0.04%, actual). The reactor was heated to a temperature between 75-85° C. (78.5° C., actual), and the reaction was allowed to proceed (76.8-80.8° C.).

After 20 h, the mixture was sampled for HPLC purity analysis every 3˜6 h until the relative area % of MR84910/(MR84910+MR84915) was below 3.0% (2.2 area %, 29 h and 44 min). The mixture was then cooled to a temperature between 20-30° C. (26.0° C., actual). After cooling, purified water (585.4 kg) was added into the mixture slowly at 20-30° C. The addition of water was highly exothermic during the first 10% added, corresponding to dissolution of the potassium phosphate, and the rate of addition should be carefully controlled. The subsequent portion was not very exothermic. The mixture was stirred for 20-30 min at a temperature between 20-30° C. and then the layers were allowed to settle prior to separation. The aqueous fraction was discarded. A sodium chloride solution which was prepared from sodium chloride (87.8 kg) and purified water (585.2 kg) was added into the organic phase at 20-30° C. The mixture was stirred for 20-30 min at a temperature between 20-30° C., and then the layers were allowed to settle prior to separation. The aqueous fraction was discarded. A citric acid solution which was prepared from citric acid monohydrate (1.1 kg), sodium chloride (52.7 kg) and purified water (351.6 kg) was added into the organic phase at a temperature between 20-30° C. The mixture was stirred for 1-2 h at this temperature, and then the layers were allowed to settle prior to separation.

The organic phase was sampled for HPLC purity analysis until the area % of MR84910 fell below 0.5 area % (0.2 area %, actual). The organic phase was concentrated at T_{jacket}≤−60° C. under reduced pressure (P≤−0.08 MPa) until 4-5 vol (468-585 L) remained in the reactor. 2-MeTHF (302.7 kg) was added into the remaining mixture at a T_{jacket}≤60° C. The organic phase was concentrated at T_{jacket}≤60° C. under reduced pressure (P≤−0.08 MPa) until 4-5 vol (468-585 L) remained in the reactor. 2-Me-THF (100.9 kg) was added into the mixture at a T_{jacket}≤60° C., and then the mixture was sampled to confirm a moisture content below 0.5%. Karl-Fischer analysis revealed a moisture content of 0.3%. Acetone (463.4 kg) was added to the mixture. A solution of p-Toluenesulfonic acid monohydrate (39.3 kg, 1.0 equiv. relative to MR84915 assay) in 2-MeTHF (100.6 kg) was prepared, and 34.9 kg of the solution was added into the 5000 L reactor at a temperature between 20-30° C. over a time of no less than 5 h. A mixture containing acetone (38.5 kg) and seed crystal (0.6 kg) was added into the mixture at a temperature between 15-35° C. The slurry was maintained at a temperature between 20-30° C. for 2-3 h. The remainder of the p-Toluenesulfonic acid monohydrate solution was added to the mixture in the 5000 L reactor over a time of not less than 10 h. The mixture was then heated to a temperature between 50-60° C. (50.7° C., actual). The mixture was maintained at temperature and stirred for 1.5-2.5 h before cooling to a temperature between −15 to −5° C. (−6.8° C., actual). The slurry was stirred at this temperature, and after 8 h, the mixture was sampled every 1-3 h for assay wt % of M84915 in the supernatant until a level below 0.8% was achieved (0.8%, actual). The mixture was filtered with a Halar-lined Nutsche centrifuge, and the reactor was rinsed with acetone twice (140.2 kg and then 139.4 kg, pre-cooled to a temperature of −15 to 5° C.) and then transferred into the centrifuge to rinse the filter cake. The filter cake was dried in rotary conical dryer at T_{jacket}≤65° C., P≤−0.06 MPa until the sum of 2-MeTHF and acetone residuals were below 2.0% by GC. After the drying was completed, the jacket was cooled to 20-30° C. MR84915 Tosylate salt was obtained as an off-white solid (127.3 kg, 101.8 kg corrected for assay wt %, 80.4 assay wt %, 66.0% yield).

M.p.: 150.6-150.7° C.

¹H NMR (400 MHz, DMSO-d₆) δ ppm 1.77-1.97 (m, 2H), 1.98-2.10 (m, 1H), 2.17-2.26 (m, 1H), 2.27 (s, 3H), 2.82-3.17 (m, 9H), 3.37 (br s, 3H), 3.48-3.65 (m, 2H), 3.70-3.84 (m, 2H), 3.88-4.13 (m, 3H), 4.21 (dd, J=17.31, 8.72 Hz, 1H), 4.39-4.49 (m, 1H), 4.51-4.66 (m, 2H), 5.06-5.23 (m, 2H), 7.08-7.13 (m, 2H), 7.33-7.45 (m, 6H), 7.45-7.52 (m, 3H), 7.53-7.61 (m, 2H), 7.73-7.79 (m, 1H), 7.90-7.96 (m, 1H), 9.67 (s, 1H).

¹³C NMR (126 MHz, DMSO-d₆) δ ppm 18.1, 20.7, 21.8, 25.5, 26.2, 35.8, 46.8, 47.2, 48.2, 49.9, 56.4, 58.4, 58.7, 64.0, 66.8, 109.0, 118.5, 118.8, 124.8, 124.9, 125.4, 125.9, 126.8, 127.6, 127.9, 128.0, 128.4, 128.6, 128.8, 129.5, 136.4, 137.0, 137.6, 145.7, 148.0, 154.2, 161.2, 164.3, 165.9.

HRMS (ESI) calculated for C₃₇H₄₁ClN₇O₃: 666.2959 [M+H]⁺. Found: 666.3036.

### Example 5

### Step (f)

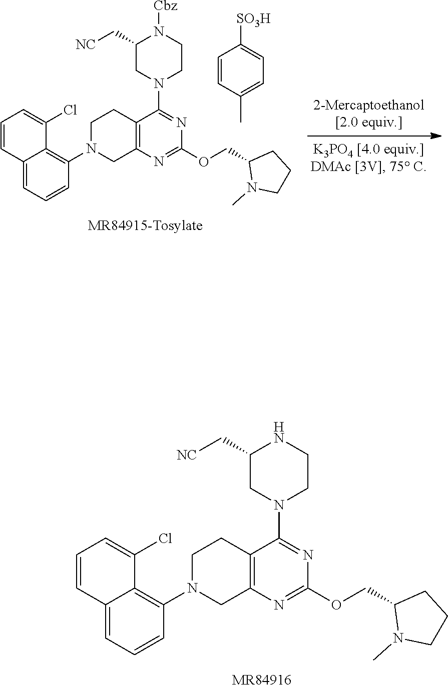

N,N-Dimethylacetamide (289.6 kg) was charged into a 3000 L glass-lined reactor and stirring was started. MR84915 Tosylate salt (123.1 kg, 99.0 kg corrected as free base, 146.8 mol, 1.0 equiv.) was added into the mixture. The reactor wall was rinsed with N,N-Dimethylacetamide (58.0 kg). Under the protection of nitrogen, anhydrous potassium phosphate (124.8 kg, 587 mol, 4 equiv.) was added. The mixture was heated to 40-50° C. (41.2° C., actual), and then nitrogen was bubbled through the mixture from the lower port of reactor at a temperature between 40-50° C. for 1-2 h. After deaeration, 2-Mercaptoethanol (23.4 kg, 294 mol, 2 equiv.) was added into mixture at 40-50° C. under the protection of nitrogen. The mixture was heated to a temperature between 75-80° C. (75.1° C., actual). The mixture was allowed to react at 70-80° C.

After 10 h, the mixture was sampled for HPLC purity analysis every 2-6 h until the area % of MR84915/(MR84915+MR84916) was below 0.5%. After 25 h and 5 min the ratio reached 0.3%. The mixture was then cooled to 20-30° C. (27.5° C., actual). Purified water (307.6 kg) was added into the mixture at a temperature equal to or less than 45° C., and then the temperature of the mixture was adjusted to 35-45° C. (37.1° C., actual). The reaction was maintained at this temperature and stirred for 0.5-1 h. Stirring was stopped and the layers were allowed to settle prior to separation. The aqueous phase was discarded, and the organic phase was kept in the reactor. The temperature of the organic phase was set to a temperature between 15-25° C. (25.0° C., actual). Purified water (86.1 kg) was added into the organic phase, and then seed crystal (0.6 kg) was added. The mixture was maintained and stirred at a temperature between 15-25° C. for 12-16 h at which point a large amount of solids were observed to precipitate. The mixture was sampled for supernatant assay wt % for informational purposes only (FIO reference value: 4.5%), and 4.1% was observed to be in the supernatant. Purified water (221.4 kg) was added into reactor at 15-25° C. The mixture was stirred at a temperature between 15-25° C. for 12-16 h, and after 8 h, the mixture was sampled every 2-4 h for supernatant assay wt % analysis until the level was below 0.7% (0.7% observed). The mixture was filtered with an agitated Nutsche filter dryer. Purified water (112.7 kg) and N,N-dimethylacetamide (70.5 kg) were added into a 3000 L glass-lined reactor, and the temperature was adjusted to between 15-25° C. The mixture was transferred into an agitated Nutsche filter dryer to rinse filter cake. Purified water (370.0 kg) and acetonitrile (28.1 kg) were added into a 3000 L glass-lined reactor, then the temperature was adjusted to 15-30° C. The filter cake was added into the water/acetonitrile mixture, and the mixture was stirred for 2-3 h at a temperature between 15-30° C. The slurry from the reactor was filtered in a centrifuge. Purified water (184.6 kg) was added into the 3000 L glass-lined reactor, and then transferred into the centrifuge to rinse the filter cake. The wet filter cake was dried in a rotary conical dryer at T_{jacket}≤45° C. until the moisture content was below 15% as judged by Karl-Fischer analysis. After drying was completed, the jacket temperature was cooled to a temperature between 20-30° C. MR84916 was obtained as a brown solid (82.0 kg, 67.8 kg corrected for assay wt %, 97.2 assay wt % on a dried basis, 85.7% yield).

M.p.: 60.3-60.4° C.

¹H NMR (400 MHz, DMSO-d₆) δ ppm 1.52-1.73 (m, 3H), 1.84-1.96 (m, 1H), 2.13 (q, J=8.67 Hz, 1H), 2.32 (d, J=1.77 Hz, 3H), 2.44-2.49 (m, 1H), 2.61-2.83 (m, 5H), 2.85-2.98 (m, 3H), 3.07 (br s, 3H), 3.37 (br s, 2H), 3.42-3.51 (m, 1H), 3.72 (s, 1H), 3.85 (br d, J=12.38 Hz, 1H) 4.01 (ddd, J=10.48, 6.69, 3.28 Hz, 1H), 4.17 (br d, J=17.43 Hz, 1H), 4.24 (dd, J=10.74, 4.93 Hz, 1H) 7.31 (ddd, J=7.58, 3.41, 0.88 Hz, 1H), 7.43 (t, J=7.83 Hz, 1H), 7.52 (t, J=7.71 Hz, 1H), 7.57 (dd, J=7.58, 1.26 Hz, 1H), 7.72 (d, J=8.08 Hz, 1H), 7.87-7.95 (m, 1H).

¹³C NMR (101 MHz, DMSO-d₆) δ ppm 21.2, 22.5, 25.6, 28.5, 41.2, 44.6, 47.6, 50.0, 51.0, 51.6, 56.9, 58.6, 63.4, 68.7, 108.2, 118.7, 118.8, 124.6, 124.9, 125.9, 126.8, 128.5, 128.8, 129.5, 137.0, 148.0, 162.1, 163.8, 165.6.

HRMS (ESI) calculated for C₂₉H₃₅ClN₇O: 532.2592 [M+H]⁺. Found: 532.2706.

### Example 6

### Step (g)

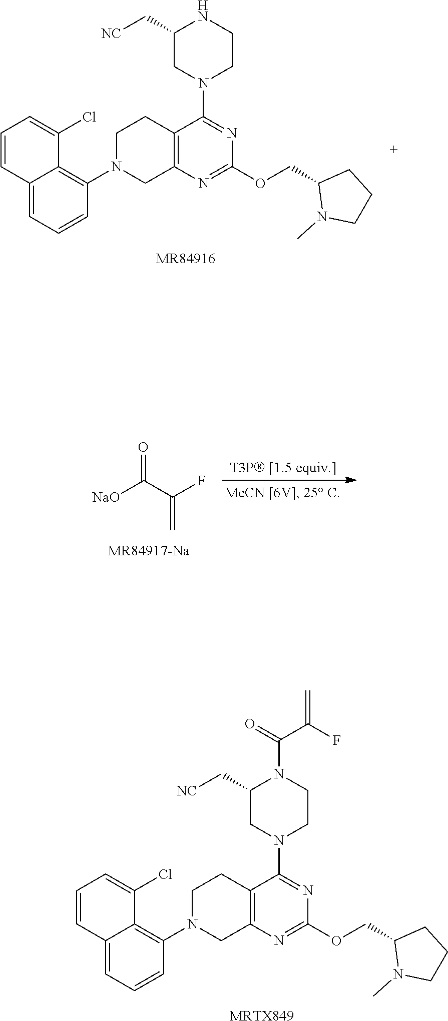

Acetonitrile (1093.0 kg) was added into a 3000 L glass-lined reactor. Next, MR84916 (81.6 kg, 68.1 kg corrected by HPLC assay wt %, 128.0 mol, 1.0 equiv.) was added to the reactor. The mixture was concentrated at a temperature below ≤45° C. under reduced pressure (P≤−0.06 MPa) until (204-272 L) 3-4 vol remained. Acetonitrile (268.0 kg) was then added into the mixture at a temperature below 45° C. The mixture was concentrated at a temperature below 45° C. under reduced pressure (P≤−0.06 MPa) until (204-272 L) 3-4 vol remained. The mixture was sampled to confirm moisture content was below 0.3% as judged by Karl-Fischer analysis (0.1%, actual). The mixture was cooled to a temperature between 10-25° C. (16.5° C., actual). Acetonitrile (163.9 kg) was added into a separate 3000 L hastelloy reactor. The mixture was sampled to confirm moisture content below 0.3% (0.02%, actual). Sodium 2-fluoroacrylate (25.0 kg, 218 mol, 1.7 equiv.) was added into the hastelloy reactor under the protection of nitrogen at a temperature between 10-20° C. It was confirmed that the sodium 2-fluoroacrylate was a finely powdered state prior to addition. The reactor wall was rinsed with acetonitrile (13.7 kg). A 50 w/w % propylphosphosphonic anhydride solution in ethyl acetate (124.7 kg, 192 mol, 1.5 equiv.) was added into the sodium 2-fluoroacrylate solution in the hastelloy reactor at a temperature between 10-20° C. under the protection of nitrogen. The mixture was stirred for not less than 2 h at a temperature between 10-20° C. The mixture containing MR84916 in the 3000 L glass-lined reactor was slowly added into the mixture containing the 2-fluoroacrylate in the 3000 L hastelloy reactor at a temperature between 10-20° C. The 3000 L glass-lined reactor containing MR84916 was rinsed with acetonitrile (18.2 kg) which was transferred into the Hastelloy reactor with the acrylate.

The reaction proceeded at 10-20° C. (14.5-18.0° C.), and after 1 h, the mixture was sampled for HPLC purity analysis every 1-3 h until the area % of MR84916/(MR84916+MRTX849) was less than 0.4% (0.3% observed at 5 h and 1 min). At a temperature between 10-30° C., the mixture was adjusted to a pH of 8-9 with a potassium carbonate solution (348.3 kg) which was prepared from potassium carbonate (41.6 kg) and purified water (307.2 kg). The mixture continued to stir for another 0.5 h and was then pH was retested for confirmation (pH 8, actual). The mixture was adjusted to a temperature of 25-35° C., stirring was stopped, and the layers were allowed to settle prior to separation. The aqueous phase was removed and kept. The phase was washed with a potassium phosphate tribasic solution which was prepared from potassium phosphate tribasic (50.1 kg) and purified water (204.4 kg) at a temperature of 25-35° C. The mixture was stirred for an additional 0.5-3 h and allowed to settle prior to separation at a temperature of 25-35° C. The aqueous phase was removed and kept. The aqueous layers were combined and extracted with 2-MeTHF (175.9 kg). The mixture was stirred for an additional 20-30 min, and the layers were allowed to settle prior to separation at a temperature between 25-35° C. The organic fractions were combined, and then the combined mixture was concentrated at a temperature ≤45° C. under reduced pressure (P≤−0.06 MPa) until (136-204 L) 2-3 vol remained. Isopropanol (429.2 kg) was added into the mixture at a temperature ≤45° C. The mixture was concentrated at a temperature ≤45° C. under reduced pressure (P≤−0.06 MPa) until (136-204 L) 2-3 vol remained. Isopropanol (320.1 kg) was added into the mixture at a temperature ≤45° C. The mixture was circulated through a CUNO filtration system. Then isopropanol (106.9 kg) was used to rinse the CUNO filter and added into the reactor. The mixture was concentrated at a temperature of 45° C. under reduced pressure (P≤−0.06 MPa) until 4.5-5.5 vol (306-374 L) remained. The mixture was sampled to confirm that residual acetonitrile residuals were less than 1.5% (0.05%, actual). The mixture was adjusted to a temperature of 33-38° C. (35.3° C., actual). Purified water (170.0 kg) was added into the mixture at 33-38° C. Form 2 seed crystal (0.2 kg) was added into the mixture at a temperature between 33-38° C. The mixture was maintained at this temperature and stirred for 2-3 h. The mixture was slowly cooled to 15-20° C. The mixture was maintained at this temperature and stirred for 6-10 h. Purified water (170.0 kg) was added into the reactor at a temperature between 15-20° C. The mixture was cooled to −3 to 7° C. slowly (4.8° C., actual). The mass was stirred at −3 to 7° C. for crystallization, and after 8 h, the mixture was sampled every 3-5 h until the mother liquor assay wt % of MRTX849 was less than 0.7% or the difference between two consecutive samples was 0.1 wt % (0.7 wt %, observed). The mixture was filtered with a stainless steel centrifuge. Purified water (102.6 kg) and isopropanol (16.4 kg) were added into a 3000 L hastelloy-lined reactor, and then transferred into a stainless steel centrifuge to rinse the filter cake. The wet filter cake was swept with nitrogen for 6-8 h, dried in a rotary conical dryer at T 40° C. until the moisture content was not more than 1% as judged by Karl-Fischer analysis. After completion of drying, the solid was cooled to 20-30° C. Isopropanol (368.4 kg) was added into a 1000 L glass-lined reactor, and then the stirrer was started. The solids from the filter cake were added to the 1000 L reactor, and the mixture was heated to a temperature between 55-60° C. (57.2° C., actual). The mixture was maintained at this temperature and stirred until the solid dissolved completely as confirmed by a visual check. The mixture was then filtered into a 1000 L hastelloy reactor (Pre-heated to Tjacket=55-60° C.) through a filtration system heated to 55-60° C. The mixture was held at 55-60° C. n-Heptane (80.5 kg) was added into the reactor, first passing through the filter for rinsing. The mixture was stirred for 0.5 h in the reactor. After the solid dissolved completely, the mixture was cooled to a temperature of 43-47° C. A seed slurry was prepared by addition of isopropanol (5.5 kg) and n-heptane (1.3 kg) into a 20 L quadri-neck flask through a capsule filter, followed by addition of Form 2 seed crystals (MRTX849 Form 2, 0.8 kg) held at a temperature between 20-25° C. The mixture was stirred until evenly mixed, and then it was recycled through a wet mill. Prior to addition of the slurry feed to the reactor, the reactor was checked to confirm full dissolution of MRTX849 and that precipitation had not occurred. After this, the Form 2 seed slurry was added into the 1000 L Hastelloy reactor at a temperature between 43-47° C. The mixture was stirred for 3-4 h at 43-47° C. The mixture was then cooled to a temperature of 28-32° C. and stirred for 4-5 h at that temperature (30.6° C., actual). After this time, the mixture was cooled to 18-22° C. and stirred for 4-5 h (20.9° C., actual). The mixture was then cooled to −3 to 7° C. (3.5° C., actual) with stirring. After 12 h, the supernatant of the mixture was sampled every 3-5 h to check the assay wt % of MRTX849 in the mother liquors, and to confirm when the level was not more than 1.2% or alternatively, when the difference between samples is equal to or less than 0.2%. During the crystallization, nitrogen was bubbled intermittently through the bottom port of the reactor. On checking the mother liquours, the assay wt % of MRTX849 was found to be 1.0%. The mixture was recycled through a wet mill at −3 to 10° C., and the batch temperature can be expected to rise by 2-3° C. during this process. The solid was sampled for particle size until the D(90) was not more than 100 m (22 μm, actual). The mixture was maintained at −3 to 7° C. for 0.5-1 h. The mixture was then filtered with a stainless steel Nutsche filter. The reactor wall was rinsed with a mixed solvent system of n-heptane (15.9 kg) and isopropanol (74.1 kg) through a liquid material filter. Then the wet mill was rinsed with these rinsing liquors, which were transferred into the reactor and then discharged into the filter to rinse the filter cake. The above operation was repeated once more with the mixed solvent of n-heptane (15.9 kg) and isopropanol (74.2 kg). The filtration was noted to be quite slow as a result of the small particle size from wet milling. The solid in the filter was swept with nitrogen at Tjacket=20-30° C. for 8-10 h, and then dried at Tjacket=35-45° C. until the isopropanol residual was not more than 6300 ppm (3488 ppm, actual) and the n-heptane residual was not more than 3500 ppm (not detected, LOD 432 ppm) as measured by GC. After drying completed, the solid was cooled to a temperature between 20-30° C. The solid was sieved until the appearance of the product was uniform and without blocking. The operation area RH % should be not more than 50%. The product (MRTX849) was obtained as an off-white solid (51.1 kg, 50.0 kg corrected for assay wt %, 100.4 assay wt %, 64.7% yield).

M.p.: 128.3-128.4° C.

¹H NMR (400 MHz, DMSO-d₆) δ ppm 1.56-1.77 (m, 3H), 1.96 (br dd, J=11.87, 7.58 Hz, 1H), 2.20 (dd, J=8.21, 2.40 Hz, 1H), 2.37 (d, J=3.54 Hz, 3H), 2.72 (br d, J=1.77 Hz, 1H), 2.91-3.03 (m, 2H), 3.04-3.23 (m, 4H), 3.28 (br dd, J=13.77, 3.66 Hz, 1H), 3.33-3.63 (m, 4H), 3.73-3.86 (m, 1H), 3.89-3.98 (m, 1H), 3.99-4.15 (m, 3H), 4.17-4.36 (m, 2H), 5.22-5.41 (m, 1H), 5.42-5.50 (m, 1H), 7.34-7.44 (m, 1H), 7.46-7.53 (m, 1H), 7.58 (q, J=7.58 Hz, 1H), 7.63 (dt, J=7.45, 1.07 Hz, 1H), 7.75-7.83 (m, 1H), 7.93-8.00 (m, 1H).

¹³C NMR (101 MHz, DMSO-d₆) δ ppm 22.5, 25.0, 25.3, 25.5, 26.8, 28.5, 41.2, 47.5, 50.0, 57.0, 58.4, 58.7, 63.4, 68.9, 99.5, 108.6, 118.1, 118.8, 124.7, 124.9, 125.9, 126.9, 128.5, 128.9, 129.5, 137.0, 148.0, 155.5 (d, J=266.39 Hz), 161.0 (d, J=11.71 Hz), 162.0, 164.3, 165.9.

¹⁹F NMR (376 MHz, DMSO-d₆) δ ppm −106.4.

HRMS (ESI) calculated for C₃₂H₃₆ClFN₇O₂: 604.2603 [M+H]⁺. Found: 604.2690.

### Example 7

### Optional Isolation of MRTX849 as Tartrate Salt

3.5 L of ethanol was added to a reactor charged with MRTX849 (875 g) and stirred until fully dissolved. In a separate reactor 1M L-tartaric acid in THF was prepared by adding 1.59 L of THF and 0.24 kg of L-tartaric acid and heated to 35-40° C. The above prepared tartaric acid solution was added to the ethanol reaction mixture of MRTX849 at 45-50° C. MRTX849 free base seed (60 mg) was added 45-50° C. and precipitate formation was slowly observed. The slurry was stirred at 45-50° C. for at least 1 h before being filtered, washed with cold ethanol, and dried in a vacuum over at 40° C. for 24 hours.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of synthesizing adagrasib, comprising either step (a) or step (a′), wherein the step (a) comprises:
a) reacting a compound of the following structure: 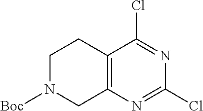 [SMILES: CC(C)(C)OC(=O)N1CCc2c(Cl)nc(Cl)nc2C1]
with a free base or a salt of a compound of the following structure: 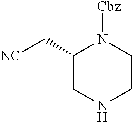 [SMILES: N#CC[C@H]1CNCCN1C(=O)OCc1ccccc1]
in the presence of a polar aprotic solvent and an organic or an inorganic base to produce a final compound of step (a) with the following structure: 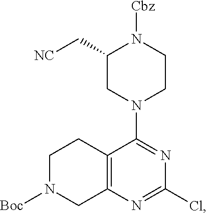 [SMILES: CC(C)(C)OC(=O)N1CCc2c(nc(Cl)nc2N2CCN(C(=O)OCc3ccccc3)[C@@H](CC#N)C2)C1]
and
wherein step (a′) comprises
a′) reacting a compound of the following structure: 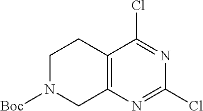 [SMILES: CC(C)(C)OC(=O)N1CCc2c(Cl)nc(Cl)nc2C1]
with the compound of the following structure: 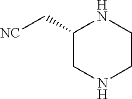
in the presence of a polar aprotic solvent and a base to produce the compound of the following structure: 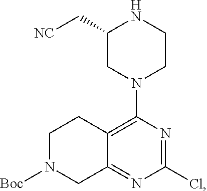 [SMILES: CC(C)(C)OC(=O)N1CCc2c(nc(Cl)nc2N2CCN[C@@H](CC#N)C2)C1]
and then
reacting this compound with benzyl chloroformate in the presence of sodium carbonate, methyl tert-butyl ether (MTBE) and water to produce a final compound of step (a′) with the following structure:

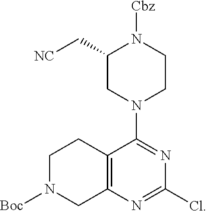 [SMILES: CC(C)(C)OC(=O)N1CCc2c(nc(Cl)nc2N2CCN(C(=O)OCc3ccccc3)[C@@H](CC#N)C2)C1]

2. The method of claim 1, further comprising step (b):
b) reacting the final compound of step (a) or step (a′) with (S)-(1-methylpyrrolidin-2-yl)methanol in the presence of a palladium catalyst, a base, a phosphorous-based ligand, and an aprotic solvent to produce a final compound of step (b) with the following structure:

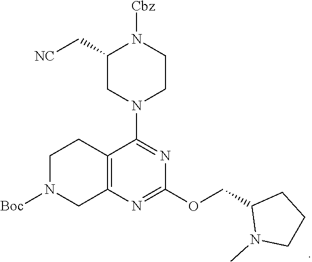

3. The method of claim 2, further comprising step (c):
c) reacting the final compound of step (b) with an acid to remove a Boc protecting group from the final compound of step (b) to produce a salt or free base of a final compound of step (c) with the following structure:

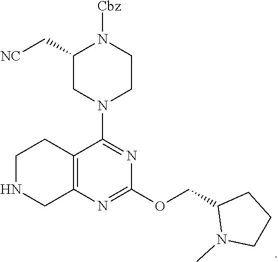

4. The method of claim 3, further comprising step (d):
d) reacting the salt or free base of the final product of step (c) with 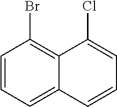 [SMILES: Clc1cccc2cccc(Br)c12]
in the presence of a palladium catalyst, a base, a phosphorous-based ligand, and an aprotic solvent to produce a final compound of step (d) with the following structure:

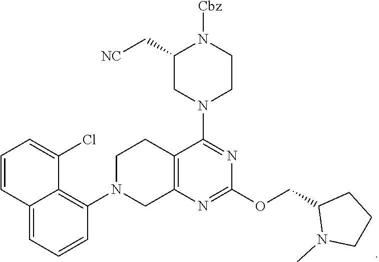

5. The method of claim 4, further comprising step (e):
e) reacting the final compound of step (d) with p-toluenesulfonic acid in the presence of a first solvent and an anti-solvent to produce a final compound of step (e) with the following structure:

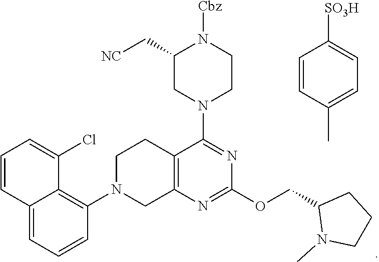

6. The method of claim 5, further comprising step (f):
f) reacting the final compound of step (e) with a thiol or thiolate in the presence of a base and a polar aprotic solvent to produce a final compound of step (f) with the following structure:

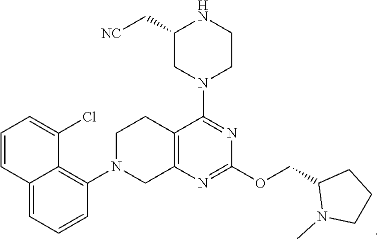

7. The method of claim 6, further comprising step (g):
(g) reacting the final compound of step (f) with 2-fluoroacrylic acid or alkali or metal salts of 2-fluoroacrylic acid with a coupling agent in the presence of a solvent and, optionally, a base to produce adagrasib.

8. A method of synthesizing adagrasib, comprising the step of reacting 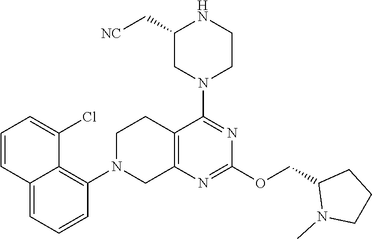
with 2-fluoroacrylic acid or alkali or metal salts of 2-fluoroacrylic acid with a coupling agent in the presence of a solvent and, optionally, a base to produce adagrasib.

9. A method of synthesizing adagrasib, comprising the steps of:
reacting 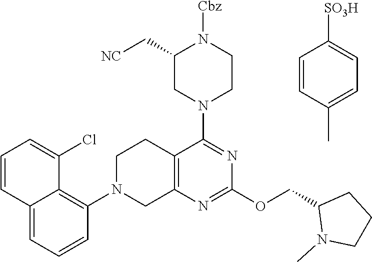
with a thiol or thiolate in the presence of a base and a polar aprotic solvent to produce 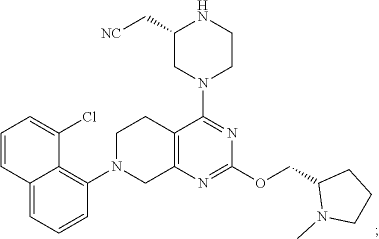
and
reacting 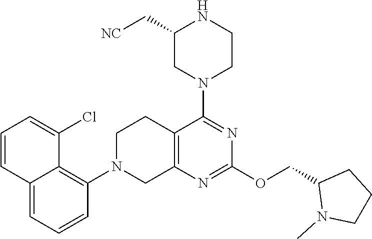
with 2-fluoroacrylic acid or alkali or metal salts of 2-fluoroacrylic acid with a coupling agent in the presence of a solvent and, optionally, a base to produce adagrasib.

10. A method of synthesizing adagrasib, comprising the steps of:
reacting 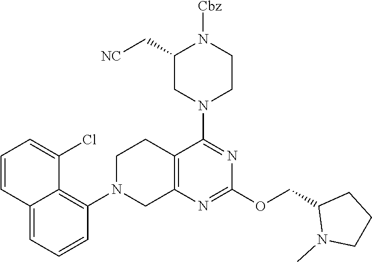
with p-toluenesulfonic acid in the presence of a first solvent and an anti-solvent to produce 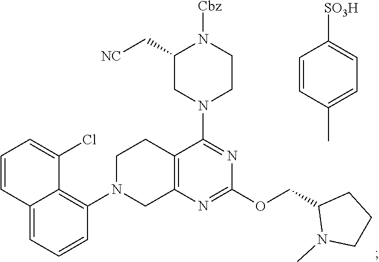
reacting 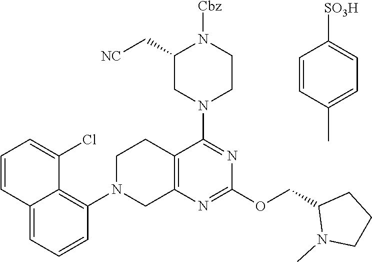
with a thiol or thiolate in the presence of a base and a polar aprotic solvent to produce 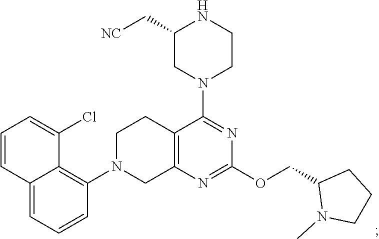
and
reacting 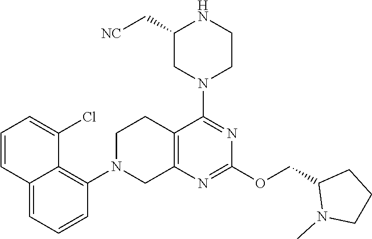
with 2-fluoroacrylic acid or alkali or metal salts of 2-fluoroacrylic acid with a coupling agent in the presence of a solvent and, optionally, a base to produce adagrasib.

11. A method of synthesizing adagrasib, comprising the steps of:
reacting the free base of 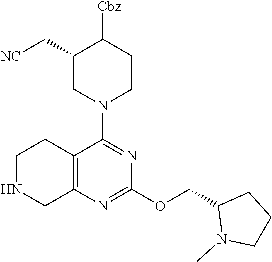
with 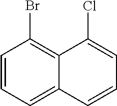 [SMILES: Clc1cccc2cccc(Br)c12]
in the presence of a palladium catalyst, a base, a phosphorous-based ligand, and an aprotic solvent to produce 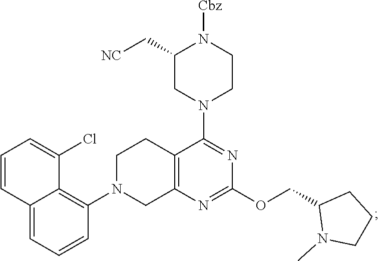
reacting 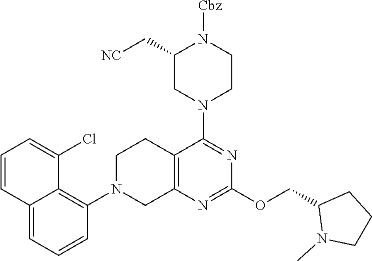
with p-toluenesulfonic acid in the presence of a first solvent and an anti-solvent to produce 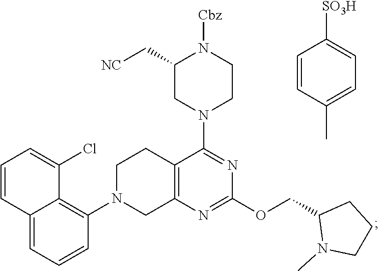
reacting 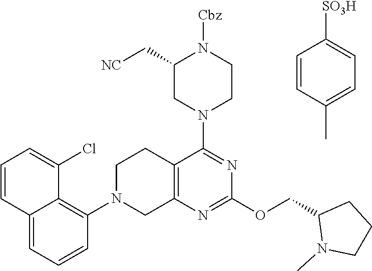
with a thiol or thiolate in the presence of a base and a polar aprotic solvent to produce 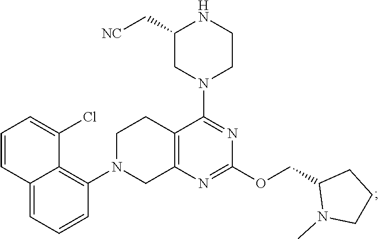
and
reacting 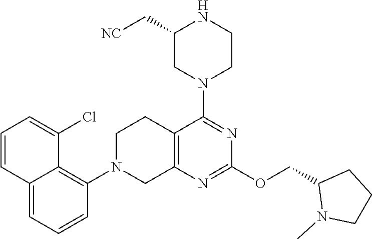
with 2-fluoroacrylic acid or alkali or metal salts of 2-fluoroacrylic acid with a coupling agent in the presence of a solvent and, optionally, a base to produce adagrasib.

12. A method of synthesizing adagrasib, comprising the steps of:
reacting 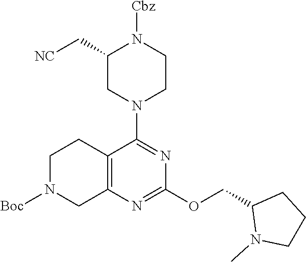
with an acid to remove a Boc protecting group to produce a salt or free base of 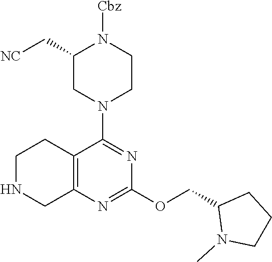
reacting the salt or free base of 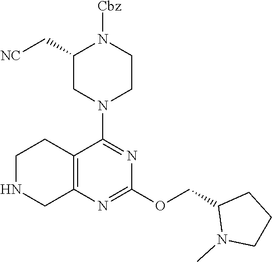
with 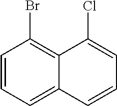 [SMILES: Clc1cccc2cccc(Br)c12]
in the presence of a palladium catalyst, a base, a phosphorous-based ligand, and an aprotic solvent to produce 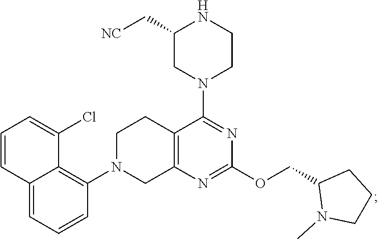
reacting 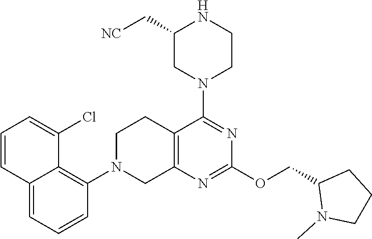
with p-toluenesulfonic acid in the presence of a first solvent and an anti-solvent to produce 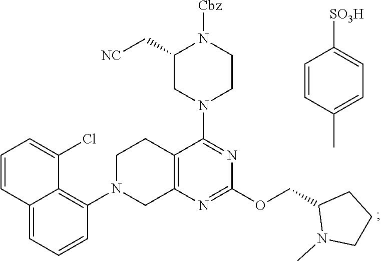
reacting 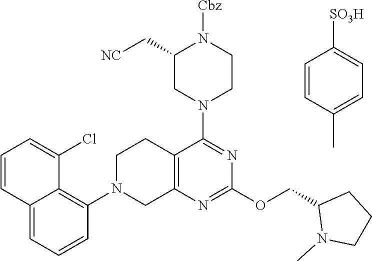
with a thiol or thiolate in the presence of a base and a polar aprotic solvent to produce 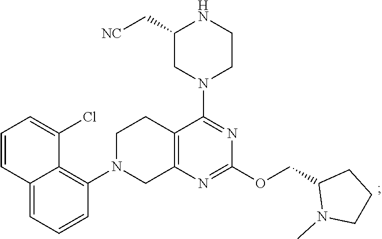
and
reacting 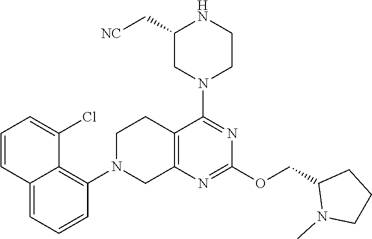
with 2-fluoroacrylic acid or alkali or metal salts of 2-fluoroacrylic acid with a coupling agent in the presence of a solvent and, optionally, a base to produce adagrasib.

13. A method of synthesizing adagrasib, comprising the steps of:
reacting 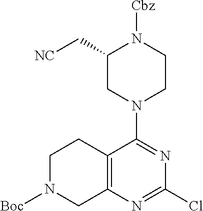 [SMILES: CC(C)(C)OC(=O)N1CCc2c(nc(Cl)nc2N2CCN(C(=O)OCc3ccccc3)[C@@H](CC#N)C2)C1]
with (S)-(1-methylpyrrolidin-2-yl)methanol in the presence of a palladium catalyst, a base, a phosphorous-based ligand, and an aprotic solvent to produce: 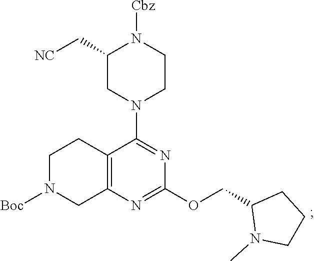
reacting 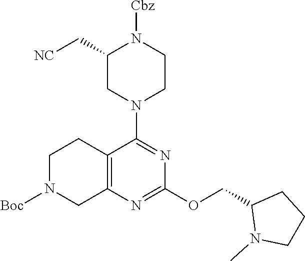
with an acid to remove a Boc protecting group to produce a salt or free base of: 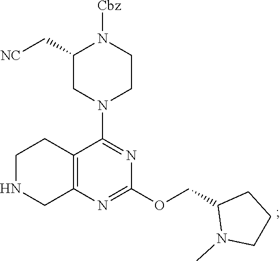
reacting the salt or free base of 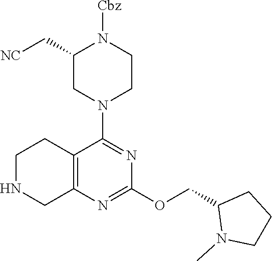
with 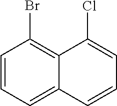 [SMILES: Clc1cccc2cccc(Br)c12]
in the presence of a palladium catalyst, a base, a phosphorous-based ligand, and an aprotic solvent to produce 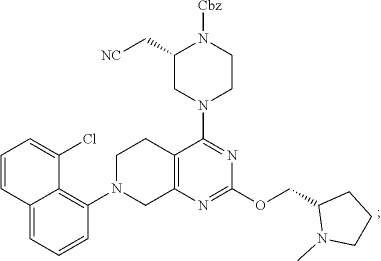
reacting 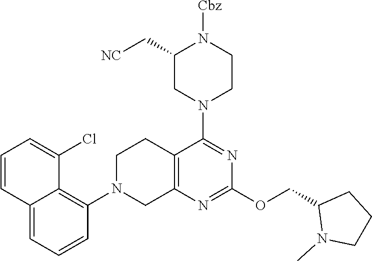
with p-toluenesulfonic acid in the presence of a first solvent and an anti-solvent to produce 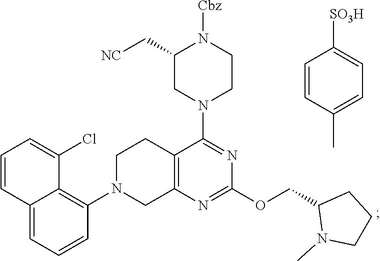
reacting 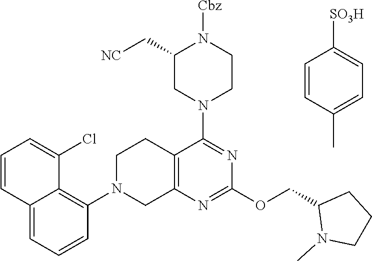
with a thiol or thiolate in the presence of a base and a polar aprotic solvent to produce 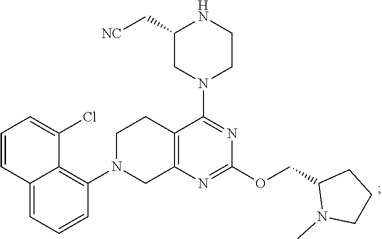
and
reacting 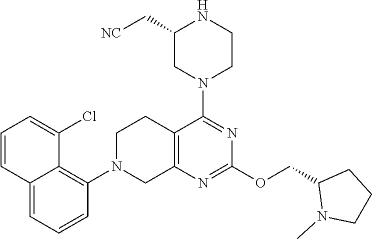
with 2-fluoroacrylic acid or alkali or metal salts of 2-fluoroacrylic acid with a coupling agent in the presence of a solvent and, optionally, a base to produce adagrasib.

14. A method of synthesizing adagrasib, comprising the steps of:
a) reacting a compound of the following structure: 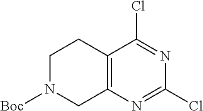 [SMILES: CC(C)(C)OC(=O)N1CCc2c(Cl)nc(Cl)nc2C1]
with a salt of a compound of the following structure: 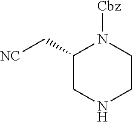 [SMILES: N#CC[C@H]1CNCCN1C(=O)OCc1ccccc1]
in the presence of a polar aprotic solvent and an organic or an inorganic base to produce a final compound of step (a) with the following structure: 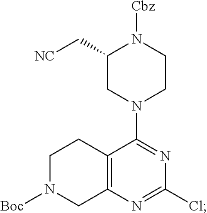 [SMILES: CC(C)(C)OC(=O)N1CCc2c(nc(Cl)nc2N2CCN(C(=O)OCc3ccccc3)[C@@H](CC#N)C2)C1]
b) reacting the final compound of step (a) with (S)-(1-methylpyrrolidin-2-yl)methanol in the presence of a palladium catalyst, a base, a phosphorous-based ligand, and an aprotic solvent to produce a final compound of step (b) with the following structure: 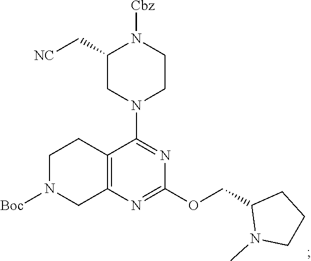
c) reacting the final compound of step (b) with an acid to remove a Boc protecting group from the final compound of step (b) to produce a salt or free base of a final compound of step (c) with the following structure: 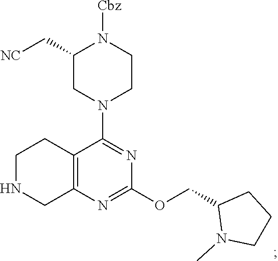
d) reacting the salt or free base of the final product of step (c) with 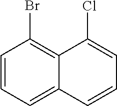 [SMILES: Clc1cccc2cccc(Br)c12]
in the presence of a palladium catalyst, a base, a phosphorous-based ligand, and an aprotic solvent to produce a final compound of step (d) with the following structure: 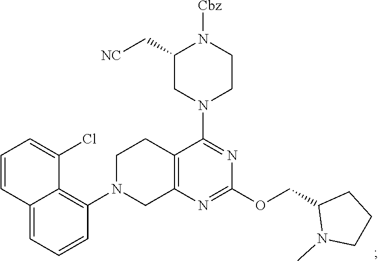
e) reacting the final compound of step (d) with p-toluenesulfonic acid in the presence of a first solvent and an anti-solvent to produce a final compound of step (e) with the following structure: 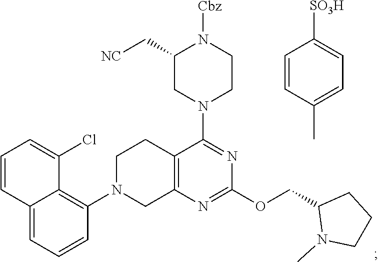
f) reacting the final compound of step (e) with a thiol or thiolate in the presence of a base and a polar aprotic solvent to produce a final compound of step (f) with the following structure: 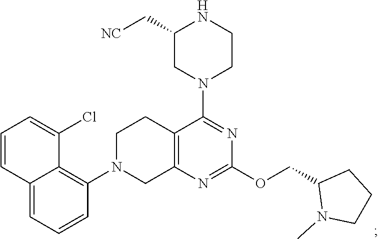
and
g) reacting the final compound of step (f) with 2-fluoroacrylic acid (or corresponding alkali or metal salts) with a coupling agent in the presence of a solvent and, optionally, a base to produce adagrasib.

15. A method of synthesizing adagrasib, comprising the steps of:
a′) reacting a compound of the following structure: 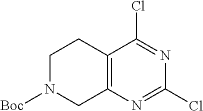 [SMILES: CC(C)(C)OC(=O)N1CCc2c(Cl)nc(Cl)nc2C1]
with the compound of the following structure: 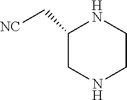
in the presence of a polar aprotic solvent and a base to produce the compound of the following structure: 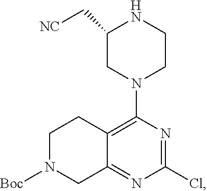 [SMILES: CC(C)(C)OC(=O)N1CCc2c(nc(Cl)nc2N2CCN[C@@H](CC#N)C2)C1]
and then
reacting this compound with benzyl chloroformate in the presence of sodium carbonate, methyl tert-butyl ether (MTBE) and water to produce a final compound of step (a′) with the following structure: 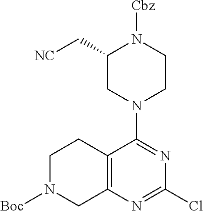 [SMILES: CC(C)(C)OC(=O)N1CCc2c(nc(Cl)nc2N2CCN(C(=O)OCc3ccccc3)[C@@H](CC#N)C2)C1]
b) reacting the final compound of step (a′) with (S)-(1-methylpyrrolidin-2-yl)methanol in the presence of a palladium catalyst, a base, a phosphorous-based ligand, and an aprotic solvent to produce a final compound of step (b) with the following structure: 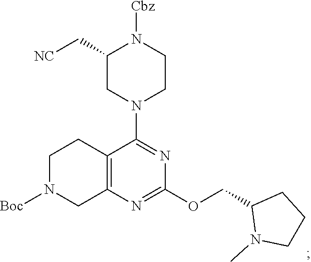
c) reacting the final compound of step (b) with an acid to remove a Boc protecting group from the final compound of step (b) to produce a salt or free base of a final compound of step (c) with the following structure: 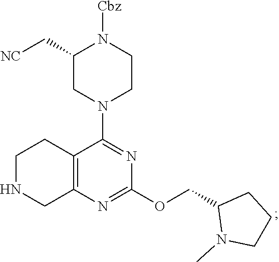
d) reacting the salt or free base of the final product of step (c) with 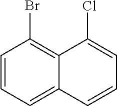 [SMILES: Clc1cccc2cccc(Br)c12]
in the presence of a palladium catalyst, a base, a phosphorous-based ligand, and an aprotic solvent to produce a final compound of step (d) with the following structure: 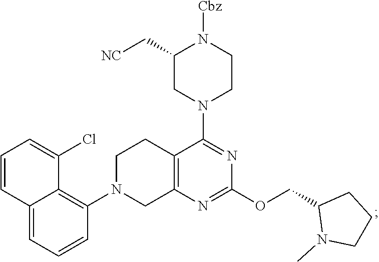
e) reacting the final compound of step (d) with p-toluenesulfonic acid in the presence of a first solvent and an anti-solvent to produce a final compound of step (e) with the following structure: 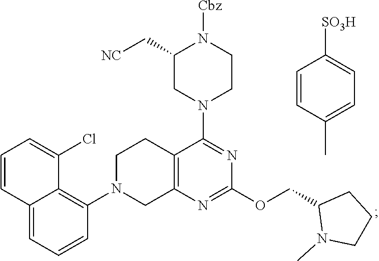
f) reacting the final compound of step (e) with a thiol or thiolate in the presence of a base and a polar aprotic solvent to produce a final compound of step (f) with the following structure: 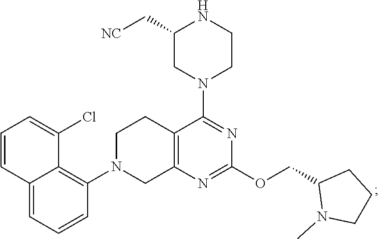
and
g) reacting the final compound of step (f) with 2-fluoroacrylic acid (or corresponding alkali or metal salts) with a coupling agent in the presence of a solvent and, optionally, a base to produce adagrasib.

16. The method of claim 14, wherein in step (a) or step (a′), the polar aprotic solvent is selected from the group consisting of dimethylacetamide (DMAc), dimethylformamide (DMF), 1,4-dioxane, tetrahydrofuran (THF), 2-methyltetrahydrofuran (2-MeTHF), acetonitrile (MeCN), dimethyl sulfoxide (DMSO), and N-methylpyrrolidone (NMP).

17. The method of claim 14, wherein in step (a) or step (a′), the polar aprotic solvent is dimethylacetamide (DMAc).

18. The method of claim 14, wherein in step (a) or step (a′), the base is an organic base.

19. The method of claim 18, wherein the organic base is selected from the group consisting of N,N-diisopropylethylamine (DIPEA), triethylamine (Et₃N), 1,4-diazabicyclo[2.2.2]octane (DABCO), and 1,8-diazabicyclo[5.4.0]undec-7-ene (DBU).

20. The method of claim 18, wherein the organic base is N,N-diisopropylethylamine (DIPEA).

21. The method of claim 14, wherein in step (a) or step (a′), the base is an inorganic base.

22. The method of claim 21, wherein the inorganic base is selected from the group consisting of carbonate, bicarbonate, and phosphate.

23. The method of claim 14, wherein in step (a) or step (a′), the salt is an organic salt.

24. The method of claim 23, wherein the organic salt is selected from the group consisting of fumarate, tartrate, malate, and citrate.

25. The method of claim 23, wherein the organic salt is a fumarate salt.

26. The method of claim 14, wherein in step (a) or step (a′), the salt is a mineral salt.

27. The method of claim 26, wherein the mineral salt is selected from the group consisting of hydrochloride, hydrobromide, sulfate and phosphate.

28. The method of claim 14, wherein step (a) is carried out at a temperature from about −10° C. to about 80° C.

29. The method of claim 14, wherein in step (b), the palladium catalyst is in the oxidation state 0 or II.

30. The method of claim 14, wherein in step (b), the palladium catalyst is selected from the group consisting of Pd₂(dba)₃, Pd(dba)₂, and Pd(OAc)₂.

31. The method of claim 14, wherein in step (b), the palladium catalyst is pre-activated.

32. The method of claim 14, wherein in step (b), the base is an organic base.

33. The method of claim 32, wherein the organic base is selected from the group consisting of N,N-diisopropylethylamine (DIPEA), triethylamine (Et₃N), 1,4-diazabicyclo[2.2.2]octane (DABCO), and 1,8-diazabicyclo[5.4.0]undec-7-ene (DBU).

34. The method of claim 14, wherein in step (b), the base is an inorganic base.

35. The method of claim 34, wherein the inorganic base is selected from the group consisting of carbonate, bicarbonate, and phosphate.

36. The method of claim 14, wherein in step (b), the phosphorous-based ligand is selected from the group consisting of a monodentate phosphorous-based ligand and a bidentate phosphorous-based ligand.

37. The method of claim 36, wherein the phosphorous-based ligand is a monodentate phosphorous-based ligand.

38. The method of claim 36, wherein the phosphorous-based ligand is a bidentate phosphorous-based ligand.

39. The method of claim 36, wherein the phosphorous-based ligand is Xanthos.

40. The method of claim 14, wherein in step (b), the aprotic solvent is selected from the group consisting of toluene, 1,4-dioxane, tetrahydrofuran (THF), 2-methyltetrahydrofuran (2-MeTHF), acetonitrile (MeCN), dimethyl sulfoxide (DMSO), and N-Methylpyrrolidone (NMP).

41. The method of claim 14, wherein step (b) is carried out at a temperature from about 20° C. to about 120° C.

42. The method of claim 14, wherein in step (c), the acid is a mineral acid.

43. The method of claim 42, wherein the mineral acid is selected from the group consisting of hydrochloric acid, sulfuric acid, nitric acid and phosphoric acid.

44. The method of claim 14, wherein step (c) is carried out at a temperature from about 20° C. to about 120° C.

45. The method of claim 14, wherein in step (d), the palladium catalyst is in the oxidation state 0 or II.

46. The method of claim 14, wherein in step (d), the palladium catalyst is selected from the group consisting of Pd₂(dba)₃, Pd(dba)₂, and Pd(OAc)₂.

47. The method of claim 14, wherein in step (d), the palladium catalyst is pre-activated.

48. The method of claim 14, wherein in step (d), the base is an organic base.

49. The method of claim 46, wherein the organic base is selected from the group consisting of DIPEA, Et₃N, DABCO, and DBU.

50. The method of claim 14, wherein in step (d), the base is an inorganic base.

51. The method of claim 50, wherein the inorganic base is selected from the group consisting of carbonate, bicarbonate, and phosphate.

52. The method of claim 14, wherein in step (d), the phosphorous-based ligand is selected from the group consisting of a monodentate phosphorous-based ligand and a bidentate phosphorous-based ligand.

53. The method of claim 52, wherein the phosphorous-based ligand is a monodentate phosphorous-based ligand.

54. The method of claim 52, wherein the phosphorous-based ligand is a bidentate phosphorous-based ligand.

55. The method of claim 14, wherein in step (d), the aprotic solvent is selected from the group consisting of toluene, 1,4-dioxane, THF, 2-MeTHF, MeCN, DMSO, and NMP.

56. The method of claim 14, wherein in step (e), the first solvent is selected from the group consisting of ketone and acetonitrile.

57. The method of claim 56, wherein the ketone is selected from the group consisting of acetone, methyl isobutyl ketone (MIBK), and methyl ethyl ketone (MEK).

58. The method of claim 14, wherein in step (e), the anti-solvent is selected from the group consisting of 2-MeTHF and or isopropyl acetate (IPAc).

59. The method of claim 14, wherein in step (f), the thiol or thiolate is selected from the group consisting of 2-mercaptoethanol, dithiothreitol (DTT), 2-(dimethylamino)ethanethiol hydrochloride, and R—SY, wherein R is selected from the group consisting of H, alkyl, and aryl, and wherein Y is selected from the group consisting of H, alkali and metal salts.

60. The method of claim 14, wherein in step (f), the base is an organic base.

61. The method of claim 60, wherein the organic base is selected from the group consisting of DIPEA, Et₃N, DABCO, and DBU.

62. The method of claim 14, wherein in step (f), the base is an inorganic base.

63. The method of claim 62, wherein the inorganic base is selected from the group consisting of carbonate, bicarbonate, and phosphate.

64. The method of claim 14, wherein in step (f), the polar aprotic solvent is selected from the group consisting of DMAc, DMF, 1,4-dioxane, THF, 2-MeTHF, MeCN, DMSO, and NMP.

65. The method of claim 14, wherein step (f) is carried out at a temperature from about 20° C. to about 120° C.

66. The method of claim 14, wherein in step (g) the solvent is selected from the group consisting of DMAc, DMF, 1,4-dioxane, THF, 2-MeTHF, MeCN, DMSO, dichloromethane (DCM), ethyl acetate (EtOAc), isopropyl acetate (IPAc), and NMP.

67. The method of claim 14, wherein in step (g), 2-fluoroacrylic acid is in the neutral form, free acid, or ionic form (as a metal or alkali salt).

68. The method of claim 14, wherein in step (g), the coupling agent is selected from the group consisting of propylphosphonic anhydride (T3P®), carbonyldiimidazole (CDI), the carbodiimide, the phosphonium, and uronium.

69. The method of claim 68, wherein the carbodiimide is selected from the group consisting of dicyclohexylcarbodiimide (DCC), diisopropylcarbodiimide (DIC), and ethyl-(N′,N′-dimethylamino)propylcarbodiimide hydrochloride (EDC·HCl).

70. The method of claim 68, wherein the phosphonium is selected from the group consisting of (benzotriazol-1-yloxy)tris(dimethylamino)phosphonium hexafluorophosphate (BOP) and (benzotriazol-1-yloxy)tripyrrolidinophosphonium hexafluorophosphate (PyBOP).

71. The method of claim 68, wherein the uronium is selected from the group consisting of O-(benzotriazol-1-yl)-N,N,N′,N′-tetramethyluronium hexafluorophosphate (HBTU) and O-(7-azabenzotriazol-1-yl)-N,N,N′,N′-tetramethyluronium hexafluorophosphate (HATU).

72. The method of claim 14, wherein in step (g), the base is an organic base.

73. The method of claim 72, wherein the organic base is selected from the group consisting of DIPEA, Et₃N, DABCO, and DBU.

74. The method of claim 14, wherein in step (g), the base is an inorganic base.

75. The method of claim 74, wherein the inorganic base is selected from the group consisting of carbonate, bicarbonate, and phosphate.

76. The method of claim 14, wherein step (g) is carried out at a temperature from about −10° C. to about 50° C.

77. A method of synthesizing adagrasib comprising the steps of:
a) reacting a compound of the following structure: 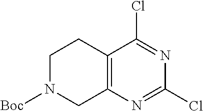 [SMILES: CC(C)(C)OC(=O)N1CCc2c(Cl)nc(Cl)nc2C1]
with a fumarate salt of a compound of the following structure: 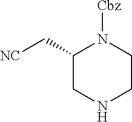 [SMILES: N#CC[C@H]1CNCCN1C(=O)OCc1ccccc1]
in the presence of DIPEA and DMAc to produce a final compound of step (a) with the following structure: 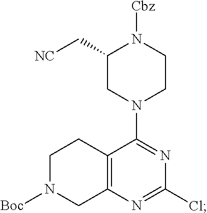 [SMILES: CC(C)(C)OC(=O)N1CCc2c(nc(Cl)nc2N2CCN(C(=O)OCc3ccccc3)[C@@H](CC#N)C2)C1]
b) reacting the final compound of step (a) with (S)-(1-methylpyrrolidin-2-yl)methanol in the presence of Pd₂(dba)₃, (R)-BINAP, K₃PO₄, and 2-MeTHF to produce a final compound of step (b) with the following structure: 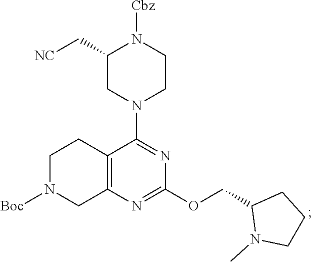
c) reacting the final compound of step (b) with a hydrochloric acid and L-tartaric acid to remove a Boc protecting group from the final compound of step (b) to produce a L-tartrate salt of a final compound of step (c) with the following structure: 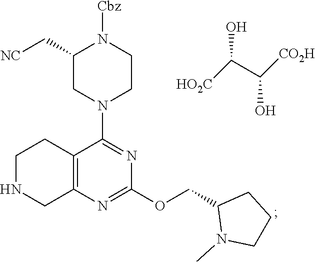
d) reacting the free base of the final product of step (c) with 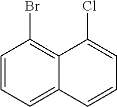 [SMILES: Clc1cccc2cccc(Br)c12]
in the presence of Pd₂(dba)₃, (R)-BINAP, K₃PO₄, and 2-MeTHF to produce a final compound of step (d) with the following structure: 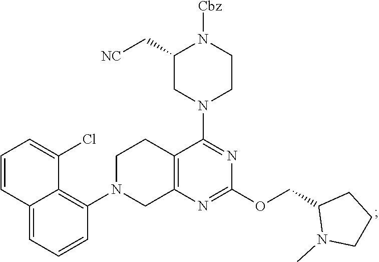
e) reacting the final compound of step (d) with p-toluenesulfonic acid in the presence of a acetone and 2-MeTHF to produce a final compound of step (e) with the following structure: 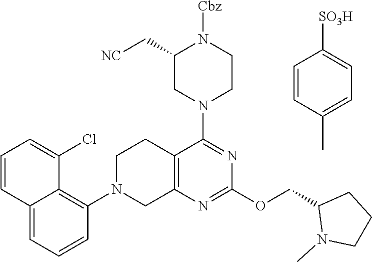
f) reacting the final compound of step (e) with 2-mercaptoethanol in the presence of K₃PO₄ and DMAc to produce a final compound of step (f) with the following structure: 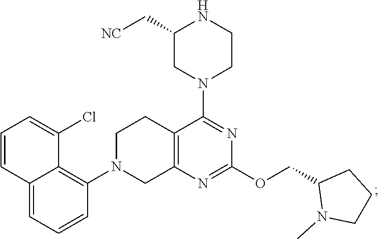
and
g) reacting the final compound of step (f) with the sodium salt of 2-fluoroacrylic acid in the presence of MeCN and propylphosphonic anhydride to produce adagrasib.

78. A method of synthesizing adagrasib comprising the steps of:
a′) reacting a compound of the following structure: 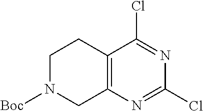 [SMILES: CC(C)(C)OC(=O)N1CCc2c(Cl)nc(Cl)nc2C1]
with a compound of the following structure: 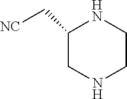
in the presence of DIPEA and DMAc to produce the compound of the following structure: 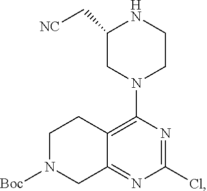 [SMILES: CC(C)(C)OC(=O)N1CCc2c(nc(Cl)nc2N2CCN[C@@H](CC#N)C2)C1]
and then
reacting this compound with benzyl chloroformate in the presence of sodium carbonate, methyl tert-butyl ether (MTBE) and water to produce a final compound of step (a′) with the following structure: 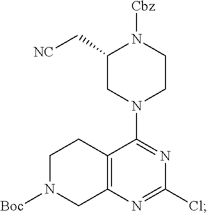 [SMILES: CC(C)(C)OC(=O)N1CCc2c(nc(Cl)nc2N2CCN(C(=O)OCc3ccccc3)[C@@H](CC#N)C2)C1]
b) reacting the final compound of step (a′) with (S)-(1-methylpyrrolidin-2-yl)methanol in the presence of Pd₂(dba)₃, Xantphos, K₃PO₄, and 2-MeTHF to produce a final compound of step (b) with the following structure: 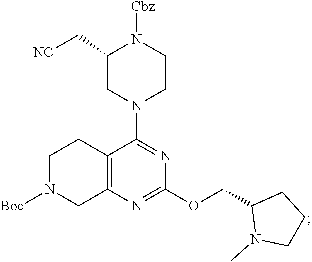
c) reacting the final compound of step (b) with a hydrochloric acid and L-tartaric acid to remove a Boc protecting group from the final compound of step (b) to produce a L-tartrate salt of a final compound of step (c) with the following structure: 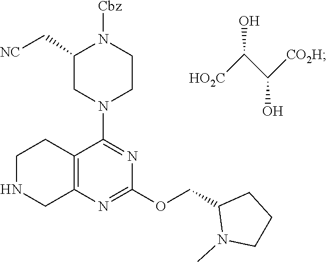
d) reacting the free base of the final product of step (c) with 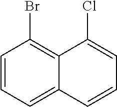 [SMILES: Clc1cccc2cccc(Br)c12]
in the presence of Pd₂(dba)₃, (R)-BINAP, K₃PO₄, and 2-MeTHF to produce a final compound of step (d) with the following structure: 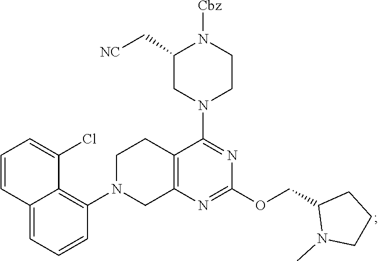
e) reacting the final compound of step (d) with p-toluenesulfonic acid in the presence of a acetone and 2-MeTHF to produce a final compound of step (e) with the following structure: 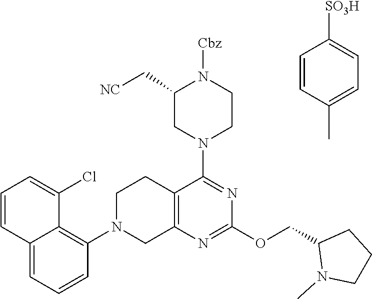
f) reacting the final compound of step (e) with 2-mercaptoethanol in the presence of K₃PO₄ and DMAc to produce a final compound of step (f) with the following structure: 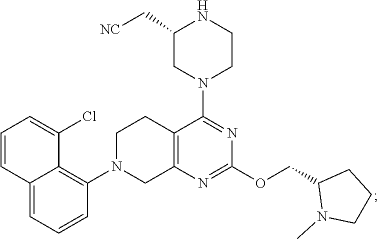
and
g) reacting the final compound of step (f) with the sodium salt of 2-fluoroacrylic acid in the presence of MeCN and propylphosphonic anhydride to produce adagrasib.

79. A compound selected from the group consisting of

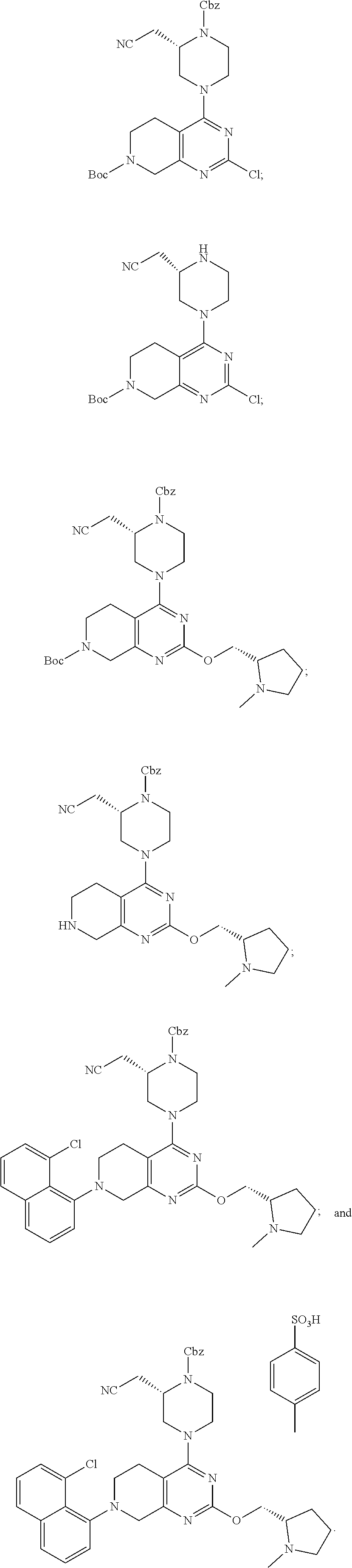

80. The compound of claim 79, wherein the compound is:

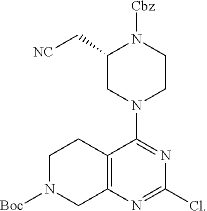 [SMILES: CC(C)(C)OC(=O)N1CCc2c(nc(Cl)nc2N2CCN(C(=O)OCc3ccccc3)[C@@H](CC#N)C2)C1]

81. The compound of claim 79, wherein the compound is

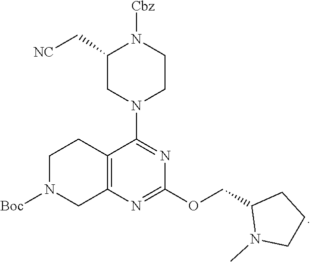

82. The compound of claim 79, wherein the compound is

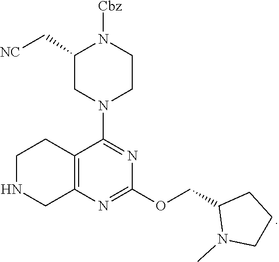

83. The compound of claim 79, wherein the compound is

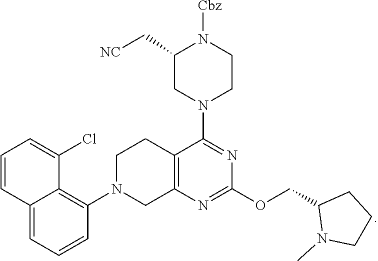

84. The compound of claim 79, wherein the compound is [chemical structure]
